(19) United States
(12) Patent Application Publication
(10) Pub. No.: US 2024/0245806 A1
(43) Pub. Date: Jul. 25, 2024

(54) TREATMENT OF NEURODEGENERATIVE DISEASE USING CSA- AND INDY-TYPE COMPOUNDS

(71) Applicant: BROWN UNIVERSITY, Providence, RI (US)

(72) Inventor: Robbert Creton, Providence, RI (US)

(21) Appl. No.: 18/562,032

(22) PCT Filed: May 27, 2022
(86) PCT No.: PCT/US2022/031241

§ 371 Date: Nov. 17, 2023

Related U.S. Application Data
(60) Provisional application No. 63/193,935, filed on May 27, 2021.

(51) Int. Cl. A61K 49/00; A61K 31/13; A61K 31/137; A61K 31/138; A61K 31/18; A61K 31/353; A61K 31/381; A61K 31/4174; A61K 31/4184; A61K 31/433; A61K 31/44; A61K 31/4439; A61K 31/47; A61K 31/4745; A61K 31/495; A61K 31/498; A61K 31/4985; A61K 31/517; A61K 31/55; A61K 31/635

(52) U.S. Cl. CPC A61K 49/0008; A61K 31/13; A61K 31/137; A61K 31/138; A61K 31/18; A61K 31/353; A61K 31/381; A61K 31/4174; A61K 31/4184; A61K 31/433; A61K 31/44; A61K 31/4439; A61K 31/47; A61K 31/4745; A61K 31/495; A61K 31/498; A61K 31/4985; A61K 31/517; A61K 31/55; A61K 31/635

(57) ABSTRACT

A method of treating or preventing a neurodegenerative disease or disorder such as Alzheimer's disease, or Down syndrome is described. The method includes administering a therapeutically effective amount of a CsA-type drug and/or an INDY-type to a subject in need thereof. Methods of identifying a neuromodulating drug using zebrafish larvae are also described.

### CROSS-REFERENCE TO RELATED APPLICATIONS

This application claims priority to U.S. Provisional Application Ser. No. 63/193,935, filed on May 27, 2021, which is hereby incorporated by reference in its entirety.

### GOVERNMENT FUNDING

This invention was made with government support under Grant Nos. RO1GM136906 and RO1EY024562. The government has certain rights in the invention.

### BACKGROUND

Drug repurposing has been proposed for treatment of various diseases, including COVID-19, cancer, neurodegenerative disorders and Alzheimer's disease. Rao et al., Aging Cell 19, e13109 (2020); Sharma et al., Life Sci 274, 119343 (2021). Repurposing is cost-effective and efficient, since existing drugs have already passed clinical trials to evaluate safety. New use may be found for FDA-approved drugs, as well as ‘failed’ drugs that passed phase I clinical trials to evaluate safety, but were not effective in phase II or III clinical trials. In this latter group, strong patent protection can provide financial incentives for additional clinical trials to examine a new use. FDA-approved drugs with expired patents may be viable as well and new use can be evaluated in population studies, before initiating additional clinical trials. Drug repurposing is a particularly powerful approach for small-molecule treatment of neural disorders. Small-molecules are more likely to pass the blood-brain barrier than protein-based biologics, which are typically excluded from the brain. In addition, many molecular targets located in visceral systems are also present in the brain. For example, angiotensin receptors in blood vessels are prime targets for blood pressure medication. These receptors are also found in neurons, astrocytes, oligodendrocytes, and microglia in the brain, indicating that angiotensin inhibitors may be reused for the treatment of neural disorders. Royea J. & Hamel, E., Geroscience 42, 1237-1256 (2020).

Alzheimer's disease has been a particular challenging disorder for drug development, with many candidate drugs failing in clinical trials. Mehta et al., Expert Opin Investig Drugs 26, 735-739 (2017). These setbacks may be caused in part by the selection of molecular targets that play a role in late neurodegenerative processes, rather than the early signaling pathways that cause Alzheimer's disease. One of the early signaling proteins that is thought to play a key role in Alzheimer's disease is the calcium-dependent serine-threonine phosphatase calcineurin. The following model has been proposed: Intracellular free calcium increases in the aging brain due to oxidative stress, mitochondrial dysfunction and amyloid R oligomers that bind transmembrane proteins. A subtle, but prolonged, increase in intracellular calcium activates calcineurin. Calcineurin dephosphorylates various signaling proteins, including the nuclear factor of activated T-cells (NFAT), BCL2-associated death protein (BAD) and glycogen synthase kinase-3 (GSK-3), which in turn induce various hallmarks of Alzheimer's disease. Reese L. & Taglialatela G., Neuropharmacol 9, 685-692 (2011). Based on this model, the inhibition of calcineurin may serve as a viable therapeutic strategy for treating neurodegenerative diseases such as early-stage Alzheimer's disease. This concept is supported by a study showing that Alzheimer's disease rarely develops in transplant patients treated with the calcineurin inhibitors cyclosporin (CsA) or tacrolimus (FK506), in all age groups above 65. Taglialatela et al., J Alzheimers Dis 47, 329-333 (2015). Small molecules that suppress the calcineurin signaling pathway in the brain, but do not suppress the immune system, would be ideal candidates for the treatment of Alzheimer's disease. But how do we find such small molecules?

Prediction of function is difficult for small molecules, especially in the brain. Small molecules often have multiple molecular targets and can affect interacting signaling pathways. In addition, small molecules may affect specific neural networks in the brain, which has approximately 100 billion neurons with 100 trillion neural connections. The analysis of behavior in animal model systems offers a solution, since subtle changes in neural function can be detected, without making assumptions on target specificity, underlying signaling pathways or the affected neurons.

The zebrafish is an emerging model system in the biomedical sciences. MacRae C. & Peterson T., Nat Rev Drug Discov 14, 721-731 (2015). Zebrafish larvae can be imaged in vivo in microplates and specific behaviors can be measured by automated image analysis. Creton, R., Behav Brain Res 203, 127-136 (2009). Moreover, high-throughput analyses of behavior have been used to screen small-molecule libraries, which led to the discovery of novel drugs with clinical relevance. Kokel et al., Nat Chem Biol 6, 231-237 (2010).

### SUMMARY OF THE INVENTION

The present invention provides a method of treating or preventing a neurodegenerative disease or disorder, comprising administering a therapeutically effective amount of a CsA-type drug to a subject in need thereof. In other embodiments, the method provides a method of treating Down Syndrome comprising administering a therapeutically effective amount of an INDY-type drug. In some embodiments, the neurodegenerative disorder is Alzheimer's disease.

These drugs described herein have all been approved by the FDA for use in treatment of various different conditions, such as high blood pressure or cancer, but have not been used for the treatment of neurodegenerative disease. By cluster analysis of behavior profiles, the inventors determined that the identified CsA-type and INDY-type drugs have an effect on neural function.

In further embodiments, a method of identifying a neuromodulating drug is provided. The method includes contacting a zebrafish larvae with a test drug; stimulating the zebrafish larvae with light and/or sound; observing the activity of the zebrafish in response to the stimulation; and characterizing the test drug as a neuromodulating drug if the activity of the zebrafish indicates an effect of the test drug on calcineurin signaling in the zebrafish larvae. In some embodiments, the method comprises identifying a CsA-type drug, while in additional embodiments the method comprises identifying an INDY-type drug.

### BRIEF DESCRIPTION OF THE FIGURES

The present invention may be more readily understood by reference to the following drawings, wherein:

FIG. 1 provides an image showing the imaging behavior in 5-day-old zebrafish larvae. The larvae are imaged in four 96-well plates for automated analysis of behavior in a 384-well format. The cropped panels on the right show three visual stimuli projected through the bottom of the plates. The red, green and blue lines move down or up in subsequent 10-minute periods. Zebrafish larvae typically swim in the same direction as the lines, called an optomotor response or OMR. Larval movements and locations are measured by automated image analysis. Inner diameter of well=7.15 mm.

FIG. 2 provides an illustration showing the analysis of behavior. Larval behaviors were examined in eighteen 10-minute periods (3 hours total) with or without stimuli. Periods 1-18 are listed in a box at the top of the Figure. Period 1-6: without visual or acoustic stimuli. Period 7-8: red lines moving down (period 7) and up (period 8). Period 9-10: green lines moving down and up. Period 11-12: blue lines moving down and up. Period 13-14: red lines moving down and up at a 16× higher speed. Period 15: without visual or acoustic stimuli. Period 16: acoustic pulses at 20-second intervals. Period 17: acoustic pulses at 1-second intervals. Period 18: acoustic pulses at 20-second intervals. Values of larval activity and location were used to calculate the following 10 parameters of behavior. 1 hr=average activity in period 1-6. P15=average activity in period 15. Hab=Habituation to acoustic stimuli at 1-second intervals. S=Startle in response to acoustic stimuli at 20-second intervals. E=Excitability in response to acoustic stimuli at 1-second intervals. R=Optomotor response (OMR) using moving red lines. G=OMR using moving green lines. B=OMR using moving blue lines. FR=OMR using red lines, moving 16× faster than all other lines. RGB=combined OMR using moving lines of any color or speed. Values are shown for untreated larvae in egg water (EW), DMSO-vehicle controls and the first 10 compounds in the library. Note the negative values for visually-guided behaviors highlighted in yellow. These larvae were treated with bromocriptine, originating from Tocris plate 1, well A3 (T1A3). The negative values indicate that larvae swim in the opposite direction as the moving visual stimuli. Activity in % move, OMR in % up. N=number of larvae.

FIG. 3 provides an illustration showing the color-coded behavioral profiles. Differences compared to the DMSO controls were color-coded by conditional formatting to provide an overview of the changes in behavior. Green=decrease more than 10%. Red=increase more than 10%. Values are shown for untreated larvae in egg water (EW), DMSO-vehicle controls and the first 10 compounds in the library. Note the decrease in activity and the increase in optomotor responses (OMR) in T1A2, which are larvae treated with UK 14,304, an adrenergic alpha-2 receptor agonist. An increase in early activity (1 hr), increase in excitability (E), and decrease in OMR was detected in T1A3, which are larvae treated with bromocriptine.

FIGS. 4A-4D provide images showing the hierarchical cluster analysis of behavioral profiles. A) Overview of all screened compounds. B) Large CsA-type cluster, containing 11 drugs that group together with the calcineurin inhibitors cyclosporine (CsA) and tacrolimus (FK506). This cluster also includes the additive inverse of proINDY, which was used as a hypothetical drug to search for inhibitors of the calcineurin (CaN)-NFAT signaling pathway. The correlation value of all 15 profiles in the CsA-type cluster is 0.86. C) Large INDY-type cluster, containing 10 drugs that cluster with the DYRK inhibitor proINDY and the additive inverse of CsA and the additive inverse of FK506 (correlation=0.86). Red box (rows 2-8)=Medium CsA cluster with CsA, FK506 and 5 other drugs (correlation=0.96). Orange box (rows 6-8)=Small CsA cluster with CsA, FK506 and XL184 (Cabozantinib), a VEGF® inhibitor (correlation=0.97). Green box (rows 13-15)=Small Inversed-INDY cluster including the additive inverse of 5 and 10 μM proINDY and Irbesartan, an angiotensin receptor antagonist (correlation=0.98). Blue box (rows 3-8)=Medium INDY cluster with 5 and 10 μM proINDY and 4 drugs (correlation=0.97). Cyan box (rows 3 & 4)=Small INDY cluster with 5 and 10 μM proINDY (correlation=0.99). Further screening or analyses may reveal additional small molecules in these classes of compounds with ‘CsA-type’ or ‘INDY-type’ behavioral profiles. Quantification of behavior: green=25% decrease, red=25% increase, black=no change, as compared to the DMSO vehicle control (% in percentage points). The identifiers refer to the location of the compounds in the small-molecule library (e.g. T1A3=Tocris plate 1, well A3). Exp=prior experiments with modulators of the calcineurin signaling pathway. In FIGS. 4B and 4C, green is shown as light grey, and red is shown as medium grey. D) Cluster analysis based on the use of artificial intelligence for automated recognition of zebrafish larvae measured a total of 25 behaviors, and identified flutamide, celecoxib, and cabergoline as drugs having CsA-type behavior profiles.

FIG. 5 provides a schematic representation model of interacting signaling pathways. Calcineurin-NFAT signaling can be suppressed using the calcineurin inhibitors cyclosporine (CsA) and tacrolimus (FK506). In contrast, proINDY activates calcineurin-NFAT signaling, by inhibiting an inhibitor (DYRK1A). The calcineurin inhibitors and proINDY have opposite effects on various behaviors, suggesting that calcineurin-NFAT signaling plays a key role in the regulation of neural function. Various lines of evidence suggest that calcineurin signaling is activated in Alzheimer's disease. Reese L. & Taglialatela, G., Curr Neuropharmacol 9, 685-692 (2011). Oxidative stress, mitochondrial dysfunction and amyloid β (Aβ) oligomers contribute to increased intracellular free calcium (Ca²⁺), which activates calcineurin. Activated calcineurin dephosphorylates various signaling proteins, such as NFAT, BAD and GSK-3, which in turn induce various hallmarks of Alzheimer's disease. This model is supported by a study showing that transplant patients on CsA and FK506 rarely develop Alzheimer's disease. Taglialatela et al., J Alzheimers Dis 47, 329-333 (2015). The inventors propose that XL184, Irbesartan and other drugs with CsA-type effects on neural function are promising candidates for the prevention and treatment of Alzheimer's disease. These seemingly unrelated drugs may act on interacting signaling pathways. VEGFR=vascular endothelial growth factor receptor. AT1=angiotensin receptor 1. XL184=Cabozantinib, PLC=phospholipase C. IP3=inositol trisphosphate, Ca²⁺=intracellular free calcium. “?”=alternative and unknown targets.

FIGS. 6A & 6B provide graphs showing measurements of zebrafish larval behavior. A) Activity in subsequent 10 minute periods (18 periods, 3 hours total). Cyclosporine (CsA) induced an increase in activity and proINDY (PI) induced a decrease in activity. B) The optomotor response (OMR); zebrafish larvae swim in the same direction as moving lines projected through the bottom of a 96-well plate. Cyclosporine induced a decrease in the optomotor response and proINDY induced an increase in the optomotor response. The legend on the right indicates how specific behavioral parameters were calculated. For example, 1-second excitability was defined as the activity in period 17 minus the activity in period in 16. Period 17a and 17b were the first and last 5 minutes in period 17. OMR Red=the response to moving red lines, defined as the OMR in period 8 minus the OMR in period 7. Fast Red; the red lines in period 13 and 14 move 16× faster than the red, green and blue lines in period 7-12. % move=the percentage of time that larvae move. % up=the percentage of time that larvae are located in the upper half of a well. Statistical analyses of activity and vision are shown in subsequent figures. N=912, 372 and 383 larvae in the DMSO vehicle control, cyclosporine and proINDY group, respectively.

FIGS. 7A & 7B provide graphs showing larval activity. A) Early activity averaged during the first hour of imaging. B) Late activity averaged during period 15. The calcineurin inhibitors cyclosporine (CsA) and tacrolimus (FK506) induced a significant increase in both early and late activity, as compared to the DMSO-vehicle controls. Rapamycin (RM) was used as a control for target specificity. RM is a macrolide immunosuppressant, similar to FK506, but inhibits mTor rather than calcineurin. RM induced a significant increase in both early and late activity, as compared to the DMSO-vehicle controls. Early activity was higher in RM-treated larvae than in FK506-treated larvae. In contrast, late activity was higher in FK506-treated larvae than in RM-treated larvae. ProINDY (PI) treated larvae displayed reduced activity, as compared to the DMSO vehicle controls; 5 μM proINDY reduced early and late activity, 10 μM proINDY reduced late activity only. Co-treatments revealed that the effects of CsA and FK506 could be rescued with either 5 or 10 μM proINDY. Conversely, the effects of 5 μM proINDY could be rescued with either CsA or FK506. Differences between corresponding groups were examined for significance using a Chi-square test with a Bonferroni correction for multiple comparisons. N=912, 372, 188, 263, 383, 275, 190, 178, 93 and 86 larvae in the 10 subsequent treatment groups. *p<0.05/14, **p<0.01/14, ***p<0.001/14.

FIGS. 8A-8D provide graphs showing optomotor response (OMR). A) Response to moving red lines. B) Response to moving green lines. C) Response to moving blue lines. D) Response to red lines, moving 16× faster than the moving lines in A-C. Larvae were treated at 5 dpf with DMSO, cyclosporine (CsA), tacrolimus (FK506), rapamycin (RM), proINDY (PI), or a combination of two treatments. Differences between corresponding groups were examined for significance using a Chi-square test with a Bonferroni correction for multiple comparisons. N=912, 372, 188, 263, 383, 275, 190, 178, 93 and 86 larvae in the 10 subsequent treatment groups. *p<0.05/14, **p<0.01/14, ***p<0.001/14.

FIGS. 9A-9D provide graphs showing responses to acoustic stimuli. A) Example of one imaging experiment analyzed during the final 40 minutes in 6-second intervals. B) Habituation to acoustic stimuli at 1-second intervals (first minus last 5 minutes of period 17). C) Startle responses (average activity in period 16 minus period 17). D) Excitability by acoustic stimuli at 1 second intervals (average activity in period 17 minus period 16). Differences between corresponding groups were examined for significance using a Chi-square test with a Bonferroni correction for multiple comparisons. N in panel A=96 larvae per treatment group. N in panel B, C and D=912, 372, 188, 263, 383, 275, 190, 178, 93 and 86 larvae in the 10 subsequent treatment groups. *p<0.05/14), **p<0.01/14), ***p<0.001/14).

FIG. 10 provides an image showing recovery of behavior in 6 and 7 day-old larvae. After treatment and initial imaging at 5 days post fertilization (dpf), zebrafish larvae were grown in egg water and imaged again at 6 and 7 dpf. Changes in behavior were calculated (Treatment—DMSO) and color coded for a visual evaluation of altered behaviors. Note that most, but not all behaviors, recovered two days after the treatment at 7 dpf. Measurements of behavior included activity in the first hour (1 hr) and period 15 (P15), optomotor responses to moving lines in red (R), green (G), blue (B), fast red (FR) and all colors and speeds combined (RGB), startle response (S), habituation (Hab) and excitability (E). *p<0.05/14, **p<0.01/14, ***p<0.001/14.

FIG. 11 provides an image showing a hierarchical cluster analysis of behavior profiles. Behaviors at 5 dpf were analyzed in Cluster 3.0 using the Euclidian distance metric with complete linkage. The clusters were then color coded in TreeView using a spectrum from green (light grey) (25% decrease) to red (medium grey) (25% increase). Three main clusters were identified, each with a distinct behavior profile: 1) the calcineurin inhibitors FK506 and CsA, 2) DMSO and rapamycin (RM), and 3) the DYRK1A inhibitor ProINDY (PI) at two concentrations. Measurements behavior include activity in the first hour (1 hr) and period 15 (P15), optomotor responses to moving lines in red (R), green (G), blue (B), fast red (FR) and all colors and speeds combined (RGB), startle response (S). habituation (Hab) and excitability (E).

FIG. 12A-12C provides an illustration of an experimental design. A) Zebrafish embryos and larvae were treated with small-molecule modulators of calcineurin signaling, from 2-3, 3-4 or 4-5 days post-fertilization (dpf). Larvae were rinsed in egg water after treatment and larval behaviors were imaged at 5 dpf. B) Zebrafish larvae were imaged in 96-well plates (inner diameter of well is 7.15 mm) with and without acoustic and visual stimuli. The image shows a red (grey) line projected through the bottom of the plate. C) The behavioral assay was divided in eighteen 10-minute periods (3 hours total). In the first 6 periods (1 hour), larvae were imaged on a light background without visual or acoustic stimuli. Larvae were then exposed to red lines moving down (period 7) and up (period 8). Larvae typically move in the same direction as the moving lines, which is referred to as an optomotor response or OMR. The optomotor response was also measured using green lines (period 9-10), blue lines (period 11-12), and ‘fast red lines’, moving 16 times faster than all other lines (period 13-14). After a period without stimuli (period 15), larvae were exposed to acoustic pulses with 20-second intervals (period 16), 1-second intervals (period 17), and 20-second intervals (period 18).

FIG. 13 provides an illustration of a model of calcineurin signaling. Elevated cytosolic free calcium (Ca²⁺) activates the serine-threonine phosphatase calcineurin. In turn, calcineurin dephosphorylates the nuclear factor of activated T-cells (NFAT), a transcription factor that plays a critical role in the immune system, heart development, neural function and behavior. The regulator of calcineurin (RCAN1) and dual-specificity tyrosine phosphorylation-regulated kinase 1A (DYRK1A) are both localized within the Down syndrome critical region on chromosome 21. In Down syndrome (trisomy 21), the extra copy of chromosome 21 leads to overexpression of RCAN1 and DYRK1A, which both suppress calcineurin-NFAT signaling. The calcineurin-NFAT signaling pathway can be modulated by small-molecule treatments. The calcineurin inhibitors cyclosporine A (CsA) and tacrolimus (FK506) suppress calcineurin-NFAT signaling. In contrast, the INhibitor of DYRK (INDY) and its cell-permeable form proINDY stimulate calcineurin-NFAT signaling, via the inhibition of an inhibitor. DYRK inhibitors have been proposed as potential therapeutics to restore suppressed calcineurin-NFAT signaling in Down syndrome. Note: RCAN1 was previously called the Down syndrome critical region 1 (DSCR1). NFAT, DYRK and RCAN in mammals are named nfat, dyrk, rcan (genes) or Nfat, Dyrk, Rcan (proteins) in zebrafish.

FIGS. 14A and 14B provide graphs of activity in 5-day-old larvae. A) Early larval activity during the first hour of imaging. B) Late activity during period 15. Activity is measured as the percentage of times that a larval zebrafish moves within a 6-second period. Differences as compared to the DMSO vehicle control are calculated for each treatment group (percentage point change). Positive numbers indicate an increase in activity, negative numbers indicate a decrease in activity (0=no change in comparison to the DMSO controls). For a complete rescue, co-treatments should be different from CsA or FK506 alone (red or green asterisks) and should be similar to the DMSO control (no black asterisks). The effects of specific treatments were tested for significance using the non-parametric Chi-square test with a Bonferroni correction for multiple comparisons. Error bars represent the standard error of the mean. *p<5.6×10⁻³ (0.05/9), **p<1.1×10⁻³ (0.01/9), ***p<1.1×10⁻⁴ (0.001/9). N=712, 207, 151, 179, 217, 163 and 232 larvae in the 2-3 dpf treatments (left to right), 489, 202, 191, 96, 238, 141 and 95 larvae in the 3-4 dpf treatments, and 760, 188, 288, 285, 285, 283 and 192 larvae in the 4-5 dpf treatments.

FIGS. 15A-15H provide graphs of the responses of 5-day-old zebrafish larvae to acoustic and visual stimuli. A) Habituation to acoustic stimuli with 1-second intervals during period 17. B) Startle response to acoustic stimuli with 20-second intervals. C) Excitability in response to acoustic stimuli with 1-second intervals. D) Optomotor response using moving red lines (OMR red). E) Optomotor response using moving green lines (OMR green). F) Optomotor response using moving blue lines (OMR blue). G) Optomotor response using red lines that move 16× faster than all other lines (OMR fast red). H) Combined optomotor responses to lines of any color and speed (OMR RGB). Differences as compared to the vehicle control are calculated for each treatment group (percentage point change). In a complete rescue, co-treatments are different from CsA or FK506 alone (red or green asterisks) and are similar to the DMSO control (no black asterisks). Effects of specific treatments were tested for significance using the non-parametric Chi-square test with a Bonferroni correction for multiple comparisons (9 comparisons). Error bars=standard error of the mean. *p<5.6×10⁻³ (0.05/9), **p<1.1×10⁻³ (0.01/9), ***p<1.1×10⁻⁴ (0.001/9). N=712, 207, 151, 179, 217, 163 and 232 larvae in the 2-3 dpf treatments (left to right), 489, 202, 191, 96, 238, 141 and 95 larvae in the 3-4 dpf treatments, and 760, 188, 288, 285, 285, 283 and 192 larvae in the 4-5 dpf treatments.

FIG. 16 provides behavioral profiles. The 10 measures of behavior, 7 treatment groups and 3 exposure periods were summarized in the panels, providing an overview of the changes in behavior. Each row is referred to as a ‘behavioral profile’, a series of changed behaviors associated with a specific treatment. Changes in comparison to the DMSO vehicle controls are shown in percentage points. Numbers having a magnitude of 20 or more in the co-treatments indicate that proINDY (PI) did not rescue the behavioral phenotype induced by cyclosporine (CsA) and tacrolimus (FK506).

### DETAILED DESCRIPTION OF THE INVENTION

The present invention provides a method of treating or preventing a neurodegenerative disease or disorder such as Alzheimer's disease, or Down Syndrome. The method includes administering a therapeutically effective amount of a CsA-type drug and/or an INDY-type to a subject in need thereof. Methods of identifying a neuromodulating drug using zebrafish larvae are also provided.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the neuromodulating activity of the compounds. In the context of the present invention, the term “aliphatic group” means a saturated or unsaturated linear or branched hydrocarbon group. This term is used to encompass alkyl, alkenyl, and alkynyl groups, for example.

The invention is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

A “subject,” as used herein, can be any animal, and may also be referred to as the patient. Preferably the subject is a mammal, such as a research animal (e.g., a monkey, rabbit, mouse or rat) or a domesticated farm animal (e.g., cow, goat, horse, pig) or pet (e.g., dog, cat). In some embodiments, the subject is a human.

Treat”, “treating”, and “treatment”, etc., as used herein, refer to any action decreasing the rate of aging of a subject or providing a benefit to a subject having a neurodegenerative disease, including improvement in the condition through lessening or suppression of at least one symptom, delay in progression of the disease, etc.

As used herein, the term “prevention” includes either preventing or decreasing the risk of developing a neurodegenerative disease or disorder. This includes prophylactic treatment of those having an enhanced risk of developing a neurodegenerative disease or disorder. An elevated risk represents an above-average risk that a subject will develop a neurodegenerative disease or disorder, which can be determined, for example, through family history or the detection of genes causing a predisposition to develop a neurodegenerative disease or disorder. A subject can also have an increased risk of developing a neurodegenerative disease or disorder as a result of injury, exposure to toxins, or infection.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect.

### CsA-Type Compounds

The inventors found that the calcineurin inhibitors cyclosporine (CsA) and tacrolimus (FK506) form a part of a large functional cluster with a number of additional seemingly unrelated drugs. Examples of these drugs include: 1) Bromocriptine, a non-selective dopamine agonist, 2) Tetrabenazine, a vesicular monoamine transporter inhibitor, 3) Rosiglitazone, a PPAR-gamma receptor agonist, 4) Nebivolol, an adrenergic beta-1 receptor antagonist or ‘beta-blocker’, 5) Sorafenib, a Raf kinase inhibitor, 6) XL184, an inhibitor of the vascular endothelial growth factor receptor, 7) Tamoxifen, a modulator of estrogen and related receptors, 8) Meclizine, a Pregnane X receptor agonist, 9) Salmeterol xinafoate, an adrenergic beta-2 receptor agonist, 10) Sulfasalazine, a NF-kB/IkB inhibitor, 11) Irbesartan, an angiotensin AT1 receptor antagonist, 12) Flutamide (an androgen receptor antagonist), 13) Celecoxib (a cyclooxygenase inhibitor) and 14) Cabergoline (a non-selective dopamine agonist). Accordingly, this class of compounds is referred to herein as ‘CsA-type’ drugs, which are characterized by their effect on brain function, instead of a compound's molecular structure or previously identified target.

The inventors have identified a number of CsA-type drugs that have the ability to treat or prevent neurodegenerative disease. These CsA-type drug can be selected from the group of drugs consisting of bromocriptine, tetrabenazine, rosiglitazone, nebivolol, sorafenib, XL184, tamoxifen, meclizine, salmeterol xinafoate, sulfasalazine, irbesartan, flutamide, celecoxib, and cabergoline. In further embodiments, the CSA-type drugs can be selected from any smaller group including these compounds. For example, in some embodiments, the group may include Lapatinib and Bazedoxifene.

In some embodiments, the CsA-type drug is XL184, also known as cabozantinib or Cabometyx®. The CsA-type drugs all act as calcineurin inhibitors. In some embodiments, the CsA-type drugs are drugs that have an effect on Zebrafish (e.g., zebrafish larvae) behavior that is very similar to the effect of cyclosporine (aka, cyclosporin or cyclosporin A).

### Inhibitor of DYRK (INDY)-Type Compounds

In some embodiments, the method further comprises administering a therapeutically effective amount of an inhibitor DYRK (INDY) type drug to the subject. DYRK1A (dual-specificity tyrosine-(Y)-phosphorylation-regulated kinase 1A) is a serine/threonine kinase essential for brain development and function, and is also known as the Down syndrome-related kinase. Small molecule INDY-type compounds have been proposed as potential therapeutics for Down syndrome. INDY-type compounds are functionally similar to ProINDY, which activates nuclear factor of activated T-cells (NFAT) via the inhibition of an inhibitor (DYRK1A) and induces behaviors that are nearly opposite to the CsA-induced behaviors. ProINDY is a prodrug of INDY, Accordingly, another aspect of the invention provides a method of treating or preventing a neurodegenerative disease, comprising administering a therapeutically effective amount of an inhibitor DYRK (INDY) type drug to a subject in need thereof. The structure of ProINDY is shown below:

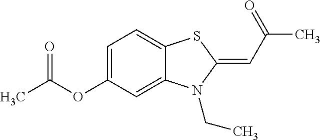 [SMILES: CCN1/C(=C/C(C)=O)Sc2ccc(OC(C)=O)cc21]

In some embodiments, the INDY-type drug is selected from the group consisting of Lapatinib, Bazedoxifene, Rucaparib, Ibutilide, Clotrimazole, Duloxetine, Tranylcypromine, Tizanidine, Venlafaxine, and UK 14,304. In further embodiments, the INDY-type drugs can be selected from any smaller group including these compounds. For example, in some embodiments, the group may include Lapatinib and Bazedoxifene. The INDY-type drugs all act as DYRK inhibitors. In some embodiments, the INDY-type drugs are drugs that have an effect on Zebrafish (e.g., zebrafish larvae) behavior that is very similar to the effect of INDY or ProINDY.

### Methods of Treating or Preventing a Neurodegenerative Disease or Disorder, or Down Syndrome

In one aspect, the present invention provides a method of treating or preventing a neurodegenerative disease or disorder. The method includes administering a therapeutically effective amount of a neuromodulating (i.e., CsA-type or INDY-type) drug to a subject in need thereof. The CsA-type and/or INDY-type drugs can be any of the drugs described herein.

Neurodegeneration generally refers to the loss of structure or function of neurons, impairment of normal neuronal functions, and includes the death of neurons. Neurodegeneration results from various different causes including genetic mutation, mitochondrial dysfunction, and the inability to handle increasing levels of oxidative or nitrosative stress can also lead to the progression of neurodegeneration. Substantial evidence from many in vitro and in vivo studies suggests that there is a commonality of events for the progression of many neurodegenerative diseases of aging. Some of these neurodegenerative diseases include Parkinson's disease, Huntington's disease, Amyotrophic Lateral Sclerosis (ALS), multiple sclerosis, and among the most common of the neurodegenerative disorders is Alzheimer's disease (AD).

In some embodiments, the neurodegenerative disease is Alzheimer's disease. Alzheimer's disease (AD) is a chronic neurodegenerative disease that results in the loss of neurons and synapses in the cerebral cortex and certain subcortical structures, resulting in gross atrophy of the temporal lobe, parietal lobe, and parts of the frontal cortex and cingulate gyrus. Wenk G., The Journal of Clinical Psychiatry. 64 Suppl 9: 7-10 (2003). Alzheimer's disease is usually diagnosed based on the person's medical history, history from relatives, and behavioral observations. The presence of characteristic neurological and neuropsychological features and the absence of alternative conditions is supportive. Advanced medical imaging with computed tomography (CT) or magnetic resonance imaging (MRI), and with single-photon emission computed tomography (SPECT) or positron emission tomography (PET) can be used to help exclude other cerebral pathology or subtypes of dementia.

In some embodiments, a method of treating Down syndrome is provided. Down syndrome is a genetic disorder caused by the presence of all or part of a third copy of chromosome 21. It is usually associated with physical growth delays, mild to moderate intellectual disability, and characteristic facial features. Dyrk1A resides within the so-called Down Syndrome Critical Region (DSCR) of human chromosome 21. Guimera et al., Hum. Mol. Genet. 5, 1305-1310 (1996), and plays a role in the pathogenesis of Down Syndrome.

The neuromodulating compounds can be used to treat or prevent a disease or disorder. A disease is a pathological process having particular signs and symptoms, whereas a disorder is a functional impairment and a disruption to the body's normal function. A disease is distinct and can be diagnoses, whereas a disorder may be a disease, but may lack sufficient clinical evidence for a diagnosis. Disease and disorder are related terms, but can be distinguished by those skilled in the art.

### Identifying Neuromodulating Drugs Using Zebrafish Larvae

An additional aspect of the invention includes methods for identifying neuromodulating drugs, such as CsA-type drugs that are effective for treating or preventing neurodegenerative disease, or INDY-type drugs that can be used for the treatment of Down Syndrome. Potential agents suitable for testing are referred to herein as “candidate agents.” A variety of different assays can be used to identify the ability of an agent to decrease the rate of neurodegenerative. Procedures for carrying out these analyses are known to those skilled in the art, and many are described in Example 1 provided herein.

Candidate agents may also be tested in animal models. For example, the ability of test compounds to treat neurodegenerative disease can be tested in Zebrafish, as described in Example 2 herein. Results are typically compared between control animals treated with candidate agents and the control cells or littermates that did not receive treatment.

Accordingly, a further aspect of the present invention provides a method of identifying a neuromodulating drug. The method includes the steps of contacting a zebrafish larvae (or embryos) with a test drug; stimulating the zebrafish larvae with light and/or sound; observing the activity of the zebrafish in response to the stimulation; and characterizing the test drug as a neuromodulating drug if the activity of the zebrafish indicates an effect of the test drug on calcineurin or DYRK signaling in the zebrafish larvae. The zebrafish larvae or embryos can be in various development stages such as 2-3, 3-4, and 4-5 dpf. Contacting, as used herein, refers to putting the test drug in the assay system being used to carry out the method such that the drug comes in contact with the zebrafish larvae, such as being carried in solution into contact with the zebrafish larvae. The zebrafish should be contacted with an effective amount of the test drug, which is an amount sufficient to simulate the zebrafish without having toxic effects. A test drugs, as used herein, refers to a drug, and preferably an FDA-approved drug, that is being tested to determine if it has neuromodulating activity and may be a CsA-type drug or an INDY-type drug.

Neuromodulating drugs are those that have an effect on the nervous system, and in particular the brain. In some embodiments, the method comprises identifying a CsA-type drug. CsA-type drugs effect the calcineurin system in the nervous system. In additional embodiments, the method comprises identifying an INDY-type drug. INDY-type drugs effect DYRK.

In some embodiments, a plurality of zebrafish larvae are contacted with the test drug in a multi-well plate. For example, the zebrafish may be contacted in a 48 well, 96 well, 192 well, or 384 well plate. Use of multi-well plates facilitates high-throughput analysis of test drugs.

The method further includes stimulating the zebrafish larvae with light and/or sound. The light and/or sound can be constant, or can be varied, both in terms of intensity and frequency. In some embodiments, the light and/or sound are provided in a pattern. For example, different type of sound and light and specific patterns of sound and light can be provided using a PowerPoint presentation that illuminates the zebrafish larvae in the multi-well assay system. As a further example, moving lines can be displayed to stimulate an optomoter response by the zebrafish larvae.

The method also includes the step of observing the activity of the zebrafish in response to the stimulation. Images are typically recorded using a camera, which can acquire high-resolution images of the larvae on the multi-well plates. The activity is typically various forms of locomotor activity, such as swimming and startle response.

The images showing the activity of the zebrafish larvae are then analyzed using image analysis. Specific methods of image analysis are described in the examples provided herein. For example, the image analysis can be used to characterizing the test drug as a neuromodulating drug if the activity of the zebrafish indicates an effect of the test drug on calcineurin or DYRK signaling in the zebrafish larvae. More specifically, the test drug is a CsA-type drug if it has an effect on calcineurin, and an ANDY-type drug if the activity has an effect on DYRK signaling. In some embodiments, the activity comprises multiple behaviors, and characterizing the activity is carried out using cluster analysis.

### Formulation and Administration of Neuromodulating Compounds

In some embodiments, the present invention provides a method for administering one or more neuromodulating compounds (e.g., CsA-type compounds) in a pharmaceutical composition. Examples of pharmaceutical compositions include those for oral, intravenous, intramuscular, subcutaneous, or intraperitoneal administration, or any other route known to those skilled in the art, and generally involves providing a neuromodulating compound formulated together with a pharmaceutically acceptable carrier.

When preparing the compounds described herein for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

For example, the maximum tolerated dose (MTD) for neuromodulating compounds can be determined in tumor-free athymic nude mice. Agents are prepared as suspensions in sterile water containing 0.5% methylcellulose (w/v) and 0.1% Tween 80 (v/v) and administered to mice (7 animals/group) by oral gavage at doses of 0, 25, 50, 100 and 200 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

The neuromodulating compounds can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

The present invention is illustrated by the following examples. It is to be understood that the particular examples, materials, amounts, and procedures are to be interpreted broadly in accordance with the scope and spirit of the invention as set forth herein.

### EXAMPLES

### Example I: Novel Use of CsA-Type Drugs Identified by Cluster Analysis of Behavioral Profiles

In the current study, we treated larvae with 190 FDA-approved drugs and measured a broad range of behaviors, including activity, acoustic startle responses, habituation, excitability and optomotor responses. These behaviors were summarized in behavioral profiles, which were compared to behavioral profiles of zebrafish larvae treated with modulators of calcineurin signaling. The profiles were examined for similarity by hierarchical cluster analysis. This cluster analysis revealed a number of seemingly unrelated drugs with ‘CsA-type’ behavioral profiles. We propose that these drugs are prime candidates for the prevention and treatment of Alzheimer's disease.

### Materials and Methods

Zebrafish. The research project has been conducted in accordance with local and federal guidelines for ethical and humane use of animals and has been reviewed and approved by the Brown University Institutional Animal Care and Use Committee. Zebrafish (Danio rerio) embryos were collected and grown to larval stages as previously described. Pelkowski et al., Behav Brain Res 223, 135-144 (2011). Adult wild-type zebrafish are maintained at Brown University as a genetically-diverse outbred strain in a mixed male and female population. Zebrafish embryos from 0-3 days post-fertilization (dpf) and zebrafish larvae from 3-5 dpf were maintained at 28.5° C. in 2 L culture trays with egg water, containing 60 mg/L sea salt (Instant Ocean) and 0.25 mg/L methylene blue in deionized water. Embryos and larvae were kept on a 12 hour light/12 hour dark cycle and were randomly assigned to different experimental groups prior to experimental manipulation. The sex of embryos and larvae cannot be determined at such early stages because zebrafish use elusive polygenic factors for sex determination, and both males and females have juvenile ovaries between 2.5 and 4 weeks of development. Zebrafish larvae were imaged at 5 dpf when the larvae display a range of locomotor behaviors and consume nutrients available in the yolk sac. Clift et al., Zebrafish 11, 455-461 (2014). Larvae are approximately 4 mm long at the 5 dpf stage.

Pharmacological treatments. Zebrafish larvae were treated with 190 FDA-approved compounds using a Tocris small-molecule library (Tocris Bioscience, Cat. No. 7200). The library contains 10 mM stocks dissolved in dimethyl sulfoxide (DMSO), which we diluted 1000× in egg water to a 10 μM final concentration. The imaging experiments also included untreated larvae in egg water and larvae treated with 1 μl/ml DMSO as a vehicle control. The effects of FDA-approved drugs were compared to previously obtained results with 10 μM cyclosporine (CsA, Enzo Life Sciences), 1 μM tacrolimus (FK506, Enzo Life Sciences), 1 μM rapamycin (Santa Cruz Biotechnology) and 5 and 1 μM proINDY (Tocris Bioscience). Larvae were exposed at 5 dpf to treatment solutions or DMSO for a total of 6 hours. Larvae were first treated in a Petri dish for 2 hours, transferred with the treatment solution to white 96-well ProxiPlates (PerkinElmer, 6006290) for 1 hour, and then imaged in the treatment solution for 3 hours.

Imaging system. Zebrafish larvae were imaged in an imaging system that holds four 96-well plates for automated analysis of behavior in a 384-well format (FIG. 1). Briefly, the imaging system is housed in a 28.5° C. temperature-controlled cabinet where larvae in white 96-well ProxiPlates are placed onto a glass stage. Above the stage, a high-resolution camera (18-megapixel Canon EOS Rebel T6 with an EF-S 55-250 mm f/4.0-5.6 IS zoom lens) captures an image of the larvae in the four 96-well plates every 6 seconds. The camera is connected to a continuous power supply (Canon ACK-E10 AC Adapter) and controlled by a laptop computer using Canon's Remote Capture software (EOS Utility, version 3), which is included with the camera. Two small speakers (OfficeTec USB Computer Speakers Compact 2.0 System) were attached speaker-side down to the glass stage. Speakers were connected by USB to the laptop computer and set to maximum volume. Below the glass stage, a M5 LED pico projector (Aaxa Technologies) with a 900 lumens LED light source displays Microsoft PowerPoint presentations through the opaque bottom of the 96-well plates.

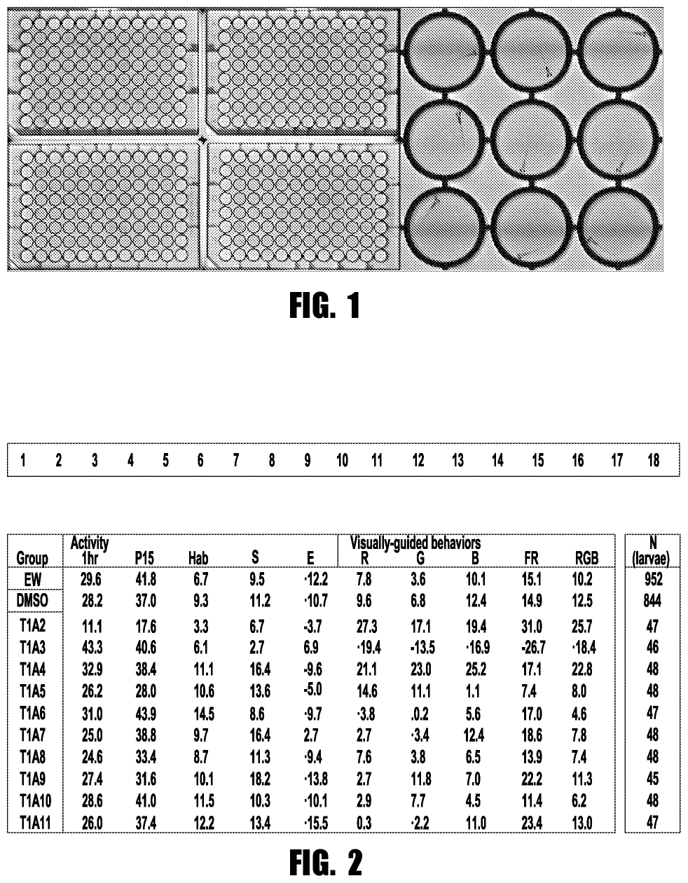

Behavioral assay. Visual and acoustic stimuli are controlled by an automated 3-hour PowerPoint presentation that is shown to the larvae. The entire 3-hour presentation has a light gray background and starts with a 1-hour period without visual or acoustic stimuli, followed by 80 minutes of visual stimuli, a 10-minute period without visual or acoustic stimuli, and 30 minutes with acoustic stimuli (FIG. 2). Larvae were not exposed to visual stimuli and acoustic stimuli at the same time.

The visual stimuli consisted of a series of moving lines that were red, green or blue. Prior studies have shown that zebrafish larvae will swim in the same direction as moving lines, a behavior that is called an optomotor response or OMR (19, 28). Our previously-developed assays for visually-guided behaviors indicate 5 dpf larvae consistently respond to 1 mm thick lines set 7 mm apart that move 7 mm per 8 seconds downwards or upwards, alternating direction in 10-minute periods (19). Additionally, the presentation included red lines that moved at a faster speed of 7 mm per 0.5 seconds (16× faster). We used the following sequence of moving visual stimuli in subsequent 10-minute periods: downward red lines, upward red lines, downward green lines, upward green lines, downward blue lines, upward blue lines, downward fast red lines, upward fast red lines. The brightness of the background (RGB=210, 210, 210), red lines (RGB=255, 0, 0), green lines (RGB=0, 180, 0), and blue lines (RGB=0, 0, 230) in the PowerPoint presentation are carefully matched to the camera settings (ISO200, Fluorescent, F5, ⅕ exposure) for optimal color separation in the automated image analysis.

The acoustic stimuli consisted of brief sine waves or ‘pulses’ (100 ms, 400 Hz) created in Audacity as 20-second sound tracks and inserted in the PowerPoint presentation. Larvae were first exposed for 10 minutes to repeated acoustic pulses with a 20-second interval, followed by 10 minutes of repeated acoustic pulses with a 1-second interval, and 10 minutes of repeated acoustic pulses with a 20-second interval.

Image analysis. We previously created an ImageJ macro for automated analysis of behavior in a 384-well format (version 26rc062019). The ImageJ macro can analyze up to four 96-well plates, with multiple treatment groups and visual stimuli that change direction, color, and speed. Users are prompted to enter information about the plates and the periods with different visual stimuli. The software opens the first image, splits the color channels, and selects a channel in which the visual stimuli and background have similar intensities. Subsequent images are subtracted from each other to remove the background and highlight larvae that move. The software then applies a threshold (40-255), selects the first well, measures the area and centroid of the larva and logs the measurements in a ‘Results’ file. This process is automatically repeated for all wells in an image and all subsequent images in a series. The frequency of larval movement and the relative position of larva in each well is analyzed and included in the Results file. The Results file of a single imaging experiment is processed in a MS Excel template (version RC012621a) and the summaries of multiple experiments are combined in a second MS Excel template (version RC013121).

Statistical analyses. Statistical analyses were performed in MS Excel as described previously. Thorn et al., Sci Rep 9, 13989 (2019) Briefly, measurements of larval movement and position were averaged in each well during 10-minute periods. The 10-minute values were then averaged between larvae in the same treatment group. Differences between experimental groups and the corresponding controls were tested for significance. We used the non-parametric Chi-square test, since larval behaviors do not follow a normal distribution.

Cluster analysis of behavioral profiles. Changes in larval activity, startle response, habituation, excitability and optomotor responses, as compared to the DMSO vehicle controls, were summarized in a ‘behavioral profile’. These behavioral profiles were generated for each compound used in this study. Similar profiles were grouped by hierarchical cluster analysis. The cluster analysis was carried out in Cluster 3.0 with an ‘Eweight’ of 0.5 for all optomotor responses, without filtering or adjusting data, and using the Euclidian distance similarity metric with complete linkage. The clusters were shown in TreeView (version 1.1.6r4) using a spectrum from green (25% decrease) to red (25% increase).

### Results

Imaging of larval behavior. Zebrafish larvae were treated with 190 compounds using a Tocris library with FDA-approved drugs and were transferred to a custom-built imaging system after a 3-hour incubation. In each imaging session, larval behaviors were recorded in four 96-well plates (FIG. 1). In total, 10,916 larvae were examined, including 952 untreated larvae, 844 DMSO-vehicle control larvae, and 9,120 (190×48) larvae treated with small-molecule drugs. Larvae that did not move throughout the experiment were automatically excluded from the analysis of behavior. While most compounds affected behavior, none of the treatments induced a complete loss of movement in all larvae. Thus, we were able to collect behavioral data for all 190 compounds. The larvae were imaged in a 3-hour behavioral assay with visual and acoustic stimuli (FIG. 2). The 3-hour assay was divided in 18 periods (10 minutes per period). In each period, values for movement and location were averaged and these averages were used to calculate the following 10 parameters of behavior: 1) activity during the first hour of imaging, 2) activity in period 15, 3) habituation to acoustic stimuli with 1-second intervals, 4) startle responses to acoustic stimuli with 20-second intervals, 5) excitability in response to acoustic stimuli with 1-second intervals, 6) optomotor responses (OMR) using moving red lines, 7) OMR using moving green lines, 8) OMR using moving blue lines, 9) OMR using red lines that move 16 times faster than all other lines, and 10) combined OMR using lines of any color or speed.

Effects of FDA-approved drugs on behavior. The FDA-approved drugs induced various specific changes in behavior (FIG. 2). FIG. 2 shows the results that were obtained with the first 10 compounds. The complete data set is included in the supplement. One treatment stood out for its striking effect on the optomotor response (FIG. 2). Larvae will normally swim in the same direction as a series of moving lines (positive OMR values). In contrast, larvae treated with bromocriptine swam in the opposite direction (negative OMR values). This behavior cannot be explained by a loss of vision, since blind fish are expected to show no response to moving lines (OMR=0).

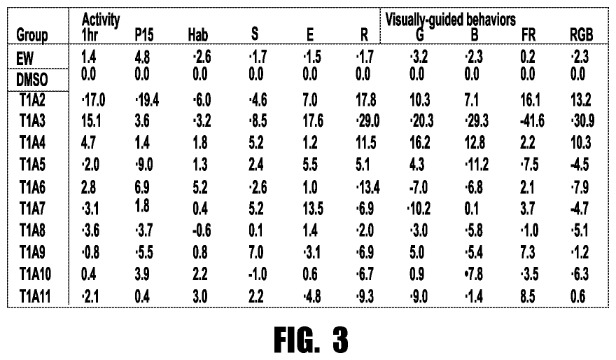

Differences in behavior compared to the DMSO-vehicle control. To create an overview of the changes in behavior, we generated ‘behavioral profiles’ by calculating differences in behavior in comparison to the DMSO-vehicle control (FIG. 3). These differences were color-coded by conditional formatting (green=10% decrease, red=10% increase). This analysis automatically highlights large changes in behavior. For example, we found a greater than 10% decrease in activity and greater than 10% increase in optomotor responses in group T1A2, which are larvae treated with UK 14,304, an adrenergic alpha-2 receptor agonist. A greater than 10% decrease in OMR was detected in group T1A3, which are larvae treated with bromocriptine.

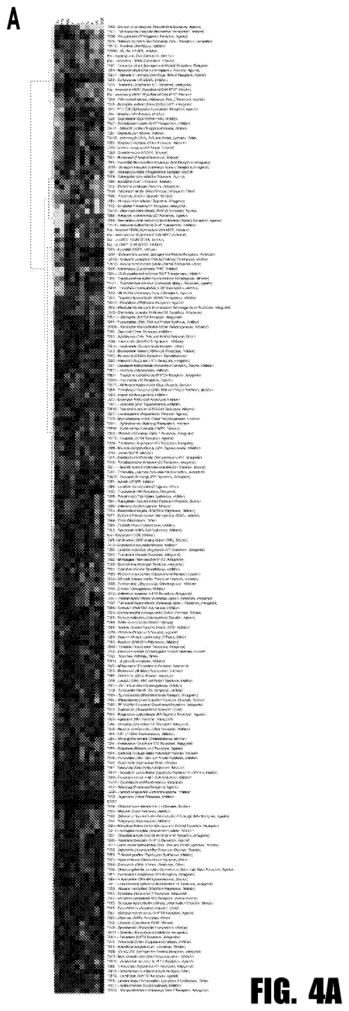

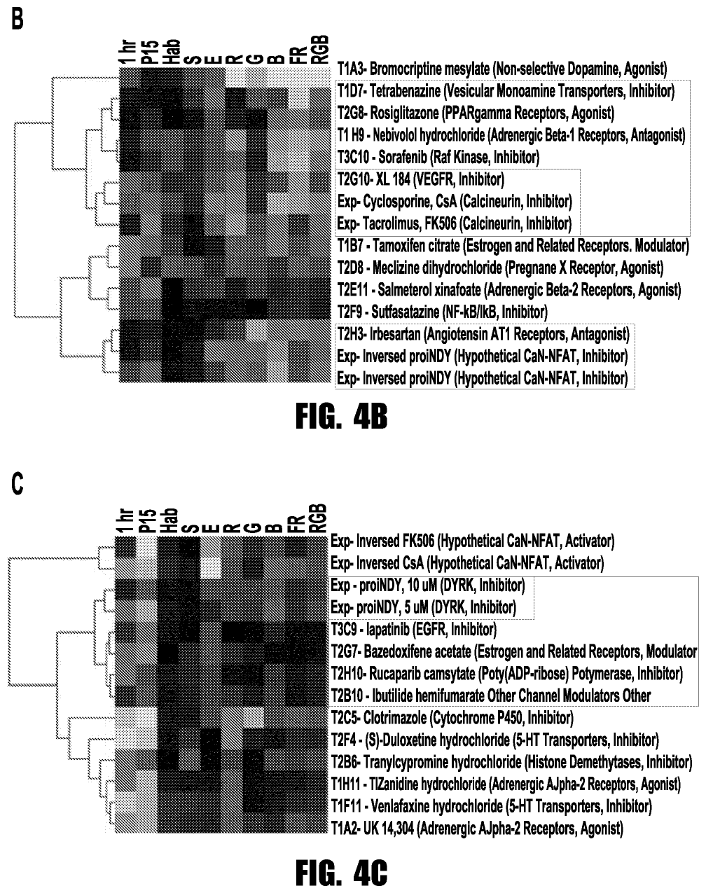

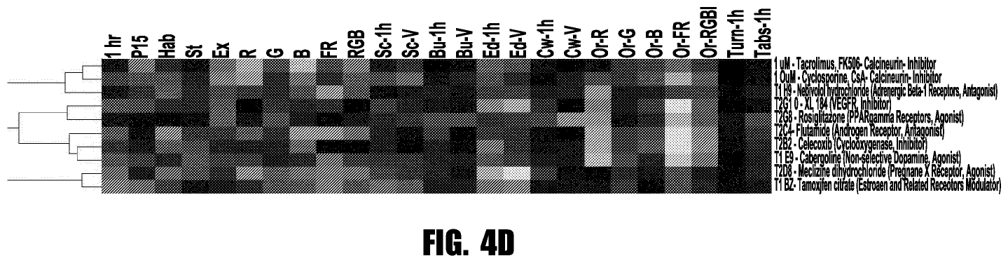

Hierarchical cluster analysis. Behavioral profiles are well suited for hierarchical cluster analysis. This analysis can reveal clusters of compounds with similar effects on behavior (FIGS. 4A-D). Behavioral profiles of compounds in the Tocris library were combined with data sets on calcineurin signaling obtained as described in Example 2.

We found that the calcineurin inhibitors cyclosporine (CsA) and tacrolimus (FK506) form a large functional cluster with 11 seemingly unrelated drugs: 1) Bromocriptine, a non-selective dopamine agonist, 2) Tetrabenazine, a vesicular monoamine transporter inhibitor, 3) Rosiglitazone, a PPAR-gamma receptor agonist, 4) Nebivolol, an adrenergic beta-1 receptor antagonist or ‘beta-blocker’, 5) Sorafenib, a Raf kinase inhibitor, 6) XL184, an inhibitor of the vascular endothelial growth factor receptor, 7) Tamoxifen, a modulator of estrogen and related receptors, 8) Meclizine, a Pregnane X receptor agonist, 9) Salmeterol xinafoate, an adrenergic beta-2 receptor agonist, 10) Sulfasalazine, a NF-kB/IkB inhibitor, 11) Irbesartan, an angiotensin AT1 receptor antagonist, Flutamide (an androgen receptor antagonist), Celecoxib (a cyclooxygenase inhibitor), and Cabergoline (a non-selective dopamine agonist). We refer to this class of compounds as ‘CsA-type’ drugs, which are characterized by their effect on brain function, instead of a compound's molecular structure or previously identified target. The behavioral profiles in this large CsA-type cluster have a correlation value of 0.86.

Within the large CsA cluster, various sub-clusters can be identified. The cluster analysis revealed a close correlation (0.96) between CsA, FK506 and the following five drugs: Tetrabenazine, Rosiglitazone, Nebivolol, Sorafenib, and XL184. A particularly tight correlation (0.97) was observed between CsA, FK506 and XL184 (Cabozantinib), which is an inhibitor of the vascular endothelial growth factor receptor (VEGFR) used for cancer treatment. Bowles et al., Drugs Today (Barc) 47, 857-868 (2011).

We also searched for CsA-type drugs that specifically inhibit calcineurin-NFAT signaling, instead of calcineurin signaling in general. For this search, we made use of previously obtained behavioral profiles induced by proINDY. ProINDY activates NFAT via the inhibition of an inhibitor (DYRK1A) and induces behaviors that are nearly opposite to the CsA-induced behaviors. We created a hypothetical NFAT inhibitor by taking the additive inverse of all proINDY-induced behaviors (+ and − are switched). We found that inversed proINDY appears within the CsA-type cluster (FIGS. 4A-D), showing that the additive-inverse approach can be used to identify drugs with CsA-type effects on the brain. Inversed proINDY clusters particularly closely with Irbesartan (correlation=0.98). Irbesartan is an angiotensin receptor antagonist used for the treatment of high blood pressure. This drug is similar to other CsA-type drugs in the observed effects on activity and optomotor responses. However, it does not increase excitability, which is considered an adverse side effect of CsA. Based on these results, we propose that Irbesartan has CsA-type effects on brain function without adverse side effects.

The cluster analysis also revealed a large group of 10 drugs (correlation=0.86) that cluster with proINDY (FIGS. 4A-D). ProINDY is an Inhibitor of DYRK, which activates calcineurin-NFAT signaling. The drugs with ‘INDY-type’ effects on neural function are: 1) Lapatinib, an EGFR inhibitor, 2) Bazedoxifene, a modulator of estrogen and related receptors, 3) Rucaparib, a poly ADP-ribose polymerase inhibitor, 4) Ibutilide, other channel modulator, 5) Clotrimazole, a cytochrome P450 inhibitor, 6) Duloxetine, a 5-HT transporter inhibitor, 7) Tranylcypromine, a histone demethylase inhibitor, 8) Tizanidine, an adrenergic alpha-2 receptor agonist, 9) Venlafaxine, a 5-HT transporter inhibitor, and 10) UK 14,304, an adrenergic alpha-2 receptor agonist. This INDY cluster also includes the additive inverse of CsA and FK506, again suggesting that the additive inverse approach can be used in the discovery of drugs with opposite effects. Overall, the ‘INDY-type’ drugs may have beneficial effects in people with Down syndrome, who have suppressed calcineurin-NFAT signaling pathways due to two genes located on chromosome 21. Ogawa et al., Nat Commun 1, 86 (2010).

### DISCUSSION

The present study shows that FDA-approved drugs can have broad-ranging effects on brain function. An intriguing result was obtained with bromocriptine, a non-selective dopamine agonist, which induced a reversed optomotor response. In the optomotor assay, larvae normally swim in the same direction as a series of moving lines. In contrast, the bromocriptine-treated larvae swim in the opposite direction. The results cannot be explained by a loss of vision, which would be expected to suppress the optomotor response, but not reverse the optomotor response. The reversed optomotor response might resemble behaviors in mice and rats infected with Toxoplasma gondii parasites. These parasites carry two genes that increase dopamine signaling and infected rodents approach stimuli that they would normally avoid. Similarly, zebrafish larvae treated with the dopamine agonist bromocriptine may approach moving visual stimuli that they would normally avoid.

Changes in behavior were summarized in behavioral profiles, which are well suited for hierarchical cluster analysis. The cluster analysis was carried out on 190 FDA-approved drugs as well as various small molecules that affect the calcineurin-NFAT signaling pathway. This signaling pathway has been described in detail in the immune system during T-cell activation, but may also play a key role in the regulation of neural function and behavior (FIG. 5). The cluster analysis identified a group of 11 FDA-approved drugs with CsA-type effects on neural function.

XL184 (Cabozantinib), displayed a particularly tight correlation (0.97) with the calcineurin inhibitors CsA and FK506. XL184 is used for cancer treatment and inhibits various receptor tyrosine kinases, including VEGFR, MET, RET, KIT, AXL and FLT3. Yakes et al., Mol Cancer Ther 10, 2298-2308 (2011). The inhibition of VEGFR, the vascular endothelial growth factor receptor, fits well within the calcineurin signaling model (FIG. 5). VEGF® acts through phospholipase C (PLC), inositol triphosphate (IP3) and calcium (Ca²⁺) release from the endoplasmic reticulum, which activates calcineurin signaling. Thus, inhibition of VEGF® likely inhibits calcineurin signaling, similar to the inhibition of calcineurin signaling with CsA or FK506. VEGF® signaling may also mediate the effects of Sorafenib, another drug in the CsA-type cluster. Sorafenib is used for cancer treatment and inhibits various protein kinases, including RAF, VEGF® and PDGFR. Ranieri et al., Curr Med Chem 19, 938-944 (2012). Thus, Sorafenib may inhibit calcium-calcineurin signaling through VEGFR, similar to XL184.

The calcineurin inhibitors CsA and FK506 induced an increase in activity and excitability, and a decrease in optomotor responses. We previously found that effects on activity and optomotor responses can be rescued by co-treatment with proINDY, suggesting that calcineurin-NFAT signaling plays a key role in the regulation of these behaviors. However, co-treatments with calcineurin inhibitors and proINDY exacerbated excitability, suggesting that excitability is an adverse side effect of calcineurin inhibitors that cannot be rescued by NFAT activation. To search for CsA-type drugs without adverse side effects, we carried out the cluster analysis using a hypothetical drug, that we named ‘inversed proINDY’. Inversed proINDY has a behavioral profile that is the additive inverse of proINDY's behavioral profile. We found that Irbesartan displayed a particularly tight correlation (0.98) with inversed proINDY. Irbesartan is an angiotensin AT1 receptor antagonist used for the treatment of high blood pressure. We found that Irbesartan increased activity and decreased optomotor responses, similar to other CsA-type drugs. However, Irbesartan did not increase excitability. Thus, our results indicate that Irbesartan is a CsA-type drug without adverse effects on excitability.

The signaling pathways affected by XL184 and Irbesartan both involve phospholipase C (FIG. 5). However, it would have been difficult to predict which drugs would fall within the CsA-type cluster based on prior knowledge of the signaling pathways. First, small-molecule drugs often affect multiple targets. For example, XL184 not only affects VEGFR, but also MET, RET, KIT, AXL and FLT3. Second, many other drugs suppress VEGFR, phospholipase C and calcium signaling, but do not induce a CsA-type behavioral profile. For example, Sunitinib and Axitinib are two VEGF® inhibitors that were included in the small-molecule screen, but did not fall in the CsA-type cluster. Third, small-molecule drugs may affect cell signaling in vitro or in specific visceral organs, but not necessarily regulate neural function in the brain. The challenges highlight the complementary roles of predictive biology and unbiased high-throughput screening, which can be used to identify small molecules with surprising effects.

Calcineurin signaling is thought to play a key role in neural degeneration and Alzheimer's disease (FIG. 5). The activation of calcineurin leads to dephosphorylation of NFAT, BAD and GSK-3, which in turn induce various hallmarks of Alzheimer's disease. This model is supported by a study showing that Alzheimer's disease rarely develops in transplant patients treated with the calcineurin inhibitors CsA or FK506, in all age groups above 65. Taglialatela et al., J Alzheimers Dis 47, 329-333 (2015). The present study identifies various FDA-approved drugs with CsA-type effects on neural function. We propose that these drugs are prime candidates for the prevention and treatment of Alzheimer's disease. These drugs are similar to the immunosuppressants CsA and FK506 in their effects on neural function, but do not suppress the immune system. In addition, it may be possible to select specific drugs or combinations of drugs that are effective in the prevention and treatment of Alzheimer's disease, without causing adverse side effects. Such studies may be pursued in Alzheimer's model mice, human population studies, and clinical trials. While the identified FDA-approved drugs have already been examined for safety, clinical trials are needed to establish the efficacy of CsA-type drugs in the prevention and treatment of Alzheimer's disease.

### Example 2: Zebrafish Model for Calcineurin-Dependent Brain Function

Calcineurin is a calcium-dependent serine-threonine phosphatase with broad clinical significance. Calcineurin inhibitors are used as immunosuppressants to prevent rejection of organ transplants. Additionally, modulated calcineurin signaling is associated with neural dysfunction in Down syndrome, Alzheimer's disease, schizophrenia, epilepsy, neuro inflammation, and traumatic brain injury. In Down syndrome (trisomy 21), the extra copy of chromosome 21 leads to overexpression of both the Down Syndrome Critical Region gene 1 (DSCR1), also called the Regulator of Calcineurin (RCAN1), and a dual-specificity tyrosine phosphorylation-regulated kinase (DYRK1A), which both suppress calcineurin signaling pathways. The suppressed calcineurin signaling pathway may affect fetal development as well as neural function later in life. People with Down syndrome frequently develop Alzheimer's disease in their fifties or sixties, although it is unclear if this is caused by a suppression of calcineurin signaling or by the gene for the Amyloid Precursor Protein (APP), which is also located on chromosome 21. In fact, various studies have shown that calcineurin signaling is elevated, rather than suppressed, in Alzheimer's disease. Reese L., & Taglialatela G., Curr Neuropharmacol 9, 685-692 (2011). The activation of calcineurin leads to dephosphorylation of various proteins, including the nuclear factor of activated T-cells (NFAT), BCL2-associated death protein (BAD) and glycogen synthase kinase-3 (GSK-3), which in turn induce various hallmarks of Alzheimer's disease, such as inflammation, cell death, and hyperphosphorylation of tau. Based on this model, the inhibition of calcineurin may serve as a viable therapeutic strategy for treating early-stage Alzheimer's disease (FIG. 5). This concept is supported by a study showing that Alzheimer's disease rarely develops in transplant patients treated with the calcineurin inhibitors cyclosporine (CsA) or tacrolimus (FK506), in all age groups above 65. Taglialatela et al., J Alzheimers Dis 47, 329-333 (2015). While some neural degeneration may be beyond repair, modulators of calcineurin signaling have the potential to prevent progressive neural degeneration in various disorders.

Little is known about the risks and potential benefits of treatments that aim to restore calcineurin signaling pathways in the brain. Clinical or population studies are limited to a few potential treatments. Animal model systems are available, but subtle morphological changes in specific neurons are easily missed when studying an organ as complex as the brain. The analysis of behavior offers a potential solution, since subtle changes in neural structure and function can be detected.

Zebrafish are well suited for large-scale analyses of behavior. Thorn et al., Sci Rep 9, 13989 (2019). Zebrafish have a prototype vertebrate brain with conserved signaling proteins such as calcineurin, Rcan, Dyrk and the nuclear factor of activated T-cells (Nfat), as well as Alzheimer's-related proteins such as the amyloid precursor protein and the microtubule-associated protein tau. Nery et al., PLoS One 9, e105862 (2014). At 5 days post-fertilization, the developing zebrafish larvae have inflated swim bladders, hunt for food and display avoidance behaviors. Colwill, R. & Creton, R., Neurosci 22, 63-73 (2011). The larvae are only 4 mm long at this time and are well suited for automated analyses of behavior in 96-well plates.

Using the zebrafish model, the present study shows that modulators of calcineurin signaling have therapeutic effects on activity and visually-guided behaviors, but adversely affect acoustic excitability. The developed methodologies provide an efficient platform for the evaluation of modulators of calcineurin signaling that restore neural function, while avoiding adverse side effects, in developmental and neurodegenerative disorders.

### Results

### Measurements of Behavior in Zebrafish Larvae

Zebrafish larvae were examined at 5 days post-fertilization (dpf) using an imaging system with four 96-well plates for automated analysis of behavior in a 384-well format (FIG. 1). The high-resolution imaging system is capable of measuring movement and location of individual larvae in each well. At 5 dpf, zebrafish larvae are approximately 4 mm long, swim freely and respond to visual and acoustic stimuli. The visual stimuli in this study consisted of moving lines (red, green or blue), projected through the bottom of opaque 96-well plates. Zebrafish larvae swim in the same direction as moving lines through their innate optomotor response or OMR. Naumann et al., Cell 167, 947-960 e920 (2016). The acoustic stimuli consisted of sound pulses at 20-second intervals or 1-second intervals. Larvae are known to display repeated startle responses to infrequent acoustic pulses at 20-second intervals, but habituate to frequent pulses at 1-second intervals.

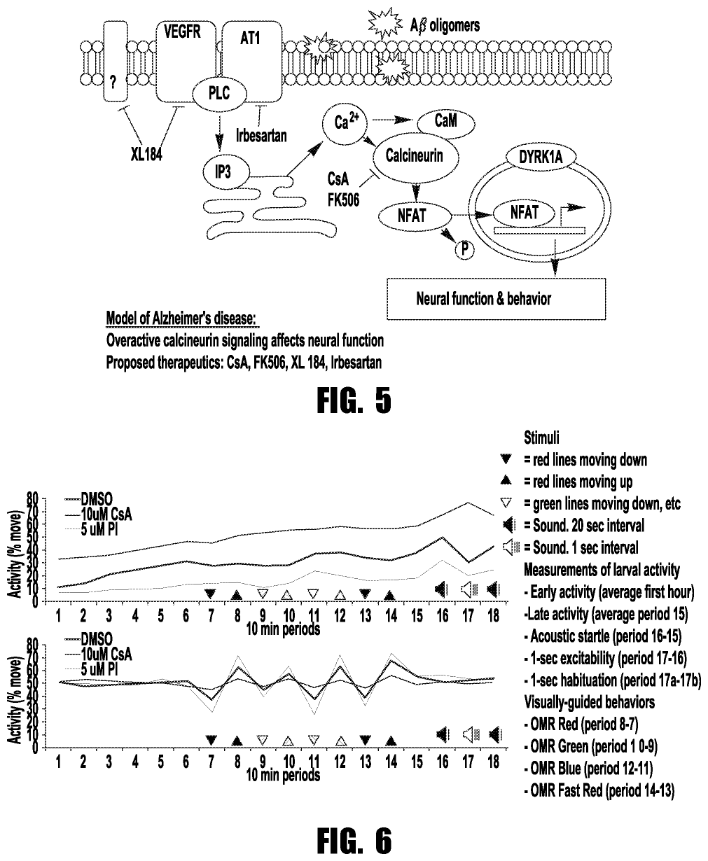

Zebrafish larvae were treated at 5 dpf with various modulators of calcineurin signaling, starting 3 hours before imaging. The larvae were then imaged for a total of 3 hours using a behavioral assay with various visual and acoustic stimuli (FIG. 6). The behavioral assay started with a 1-hour period without stimuli, followed by 80 minutes with visual stimuli, 10 minutes without visual or acoustic stimuli, and 30 minutes with acoustic stimuli. Values of activity (% move) and position (% up) were averaged in individual larvae during 10-minute periods, for a total of 18 periods. These values were subsequently averaged between larvae in the same treatment group. We examined a total of 10 treatment groups. For clarity, only 3 treatment groups were graphed (FIG. 6), but all 10 treatment groups were analyzed in detail as shown in subsequent figures. The 18-period graphs show that multiple calcineurin-sensitive behaviors can be examined in a single assay. The assay provides quantitative information on activity, visually-guided behaviors and acoustic startle responses.

### Pharmacological Treatments

To determine if larval zebrafish behavior is affected by modulation of calcineurin signaling, we imaged 5 day-old larvae in the following 10 treatment groups: 10 μM cyclosporine A (CsA), 1 μM tacrolimus (FK506), 5 or 10 μM proINDY, a combination of CsA+5 or 10 μM proINDY, or a combination of FK506+5 or 10 μM proINDY, DMSO as a vehicle control, and 1 μM rapamycin as a control for target specificity. Rapamycin and FK506 are both macrolide immunosuppressants with similar structures, however, rapamycin affects Target of Rapamycin (TOR) signaling instead of calcineurin signaling. Vellanki et al., Front Mol Biosci 7, 588913 (2020). The concentrations of CsA, FK506 and rapamycin were selected based on prior studies in zebrafish embryos and larvae. Clift et al., Behav Brain Res 282, 117-124 (2015). The two concentrations of proINDY, a membrane-permeable form of INDY (inhibitor of DYRK), were selected based on studies in cell lines and Xenopus embryos. Ogawa et al., Nat Commun 1, 86 (2010). We found that none of the treatments interfered with larval survival 1 or 2 days after treatment.

### Activity

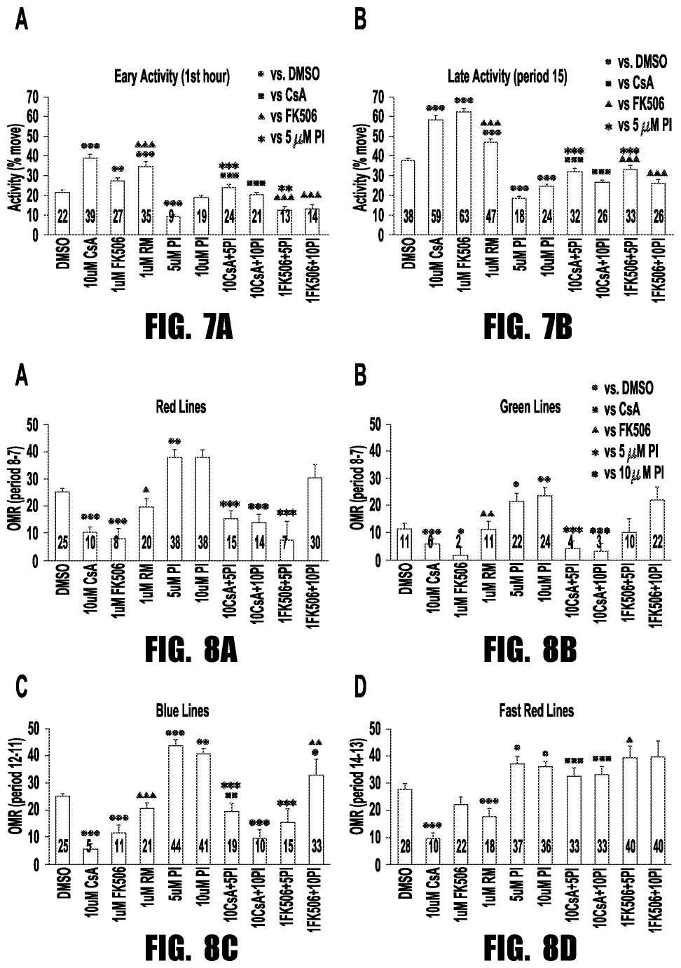

Activity was examined both early in the behavioral assay, as an average of activity during the first hour of imaging, and late in the assay, during period 15 (FIG. 7). Early and late activity values were determined when larvae were imaged without visual or acoustic stimuli. We found that the calcineurin inhibitors CsA and FK506 increased early larval activity in comparison to the DMSO control (FIG. 7a). Rapamycin also increased early activity, with activity values that exceeded activity in the DMSO control and FK506 treatment group. In contrast, proINDY treatments induced a decrease in early activity, in comparison to DMSO controls. We examined if the effects of CsA and FK506 could be rescued by the addition of proINDY and, conversely, if the effects of proINDY could be rescued by the addition of CsA and FK506. Our results show that this was indeed possible. For example, co-treatment of 10 μM CsA+5 μM proINDY induced a decrease in early activity as compared to the CsA treatment alone, and induced an increase in early activity as compared to the proINDY treatment alone.

Similar results were obtained in the analysis of late activity (FIG. 7b). CsA, FK506 and rapamycin increased activity and proINDY decreased activity in comparison to the DMSO control. In addition, we again found a rescue of activity in co-treatments of CsA+proINDY and FK506+proINDY. While the overall patterns of early and late activity were very similar in the rapamycin group and the FK506 group, we did observe a noticeable difference between the two treatments. Early activity is higher in the rapamycin group than the FK506 group (FIG. 7a), while late activity is lower in the rapamycin group than the FK506 group (FIG. 7b). Thus, the effects of FK506 and rapamycin on early and late activity were similar, but not the same. Based on the results described above, we conclude that the calcineurin-NFAT signaling pathway regulates activity in zebrafish larvae.

### Visually-Guided Behaviors

The optomotor response or OMR was examined in 5 dpf larvae using red, green and blue lines as well as red lines that move 16 times faster than all other lines. The optomotor response was calculated by subtracting the average larval position in two subsequent 10-minute periods, when lines moved down and then up (see FIG. 6). Optomotor responses were examined in all 10 treatment groups to determine if visually-guided behaviors are affected by modulation of calcineurin signaling (FIG. 8). We first analyzed the visually-guided response to red lines (FIG. 8a). Larvae treated with the calcineurin inhibitors CsA and FK506 displayed decreased optomotor responses, compared to the DMSO vehicle control. Rapamycin did not induce a significant change in the optomotor response, in comparison to the DMSO control. The optomotor response of the rapamycin treatment group was higher than the optomotor response of the FK506 treatment group. Treatment with 5 μM ProINDY led to an increased optomotor response in comparison to the DMSO control. This excessive optomotor response can be rescued by co-treatment with CsA or FK506. Similar results were obtained when analyzing responses to green lines (FIG. 8b) and blue lines (FIG. 8c). The analysis of larval responses to fast red lines (FIG. 8d) revealed a similar CsA-induced decrease in the optomotor response and proINDY-induced increase in the optomotor response. Based on the differential OMR observed across treatment groups, we conclude that the calcineurin-NFAT signaling pathway affects optomotor responses in zebrafish larvae.

### Acoustic Startle Responses

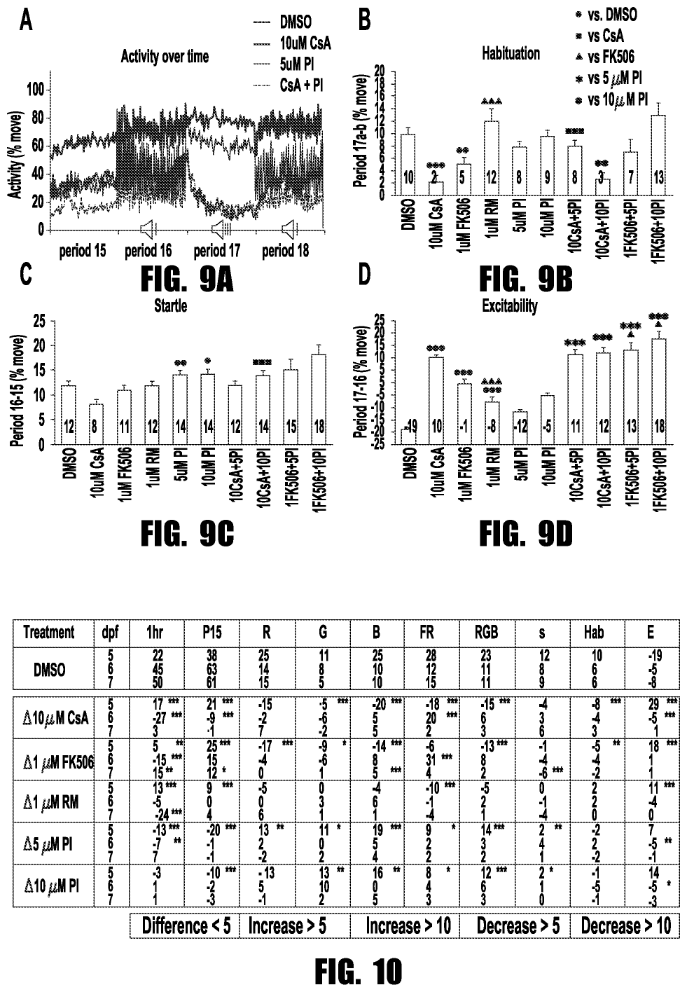

Previous studies have shown that zebrafish larvae will repeatedly startle when exposed to infrequent acoustic stimuli at 20-second intervals, but habituate to frequent acoustic stimuli at 1-second intervals. Wolman et al., Proc Natl Acad Sci USA 108, 15468-15473 (2011). We examined these startle responses in four 10-minute periods: period 15 without acoustic stimuli, period 16 with infrequent acoustic stimuli, period 17 with frequent stimuli and period 18 with infrequent stimuli (FIG. 9). As anticipated, DMSO-treated control larvae displayed stable activity during period 15, increased activity during period 16, gradually decreasing activity during period 17, and increased activity during period 18 (FIG. 9a). In contrast, the CsA-treated larvae displayed strongly-increased activity throughout period 17, which indicates that larvae lost the ability to habituate, and continuously startled in response to frequent acoustic pulses at 1-second intervals. This CsA-induced hyperexcitability was not rescued by co-treatment with proINDY. To examine these effects in more detail, we measured habituation, acoustic startle, and 1-second excitability in all 10 treatment groups. CsA and FK506-treated larvae displayed decreased habituation, as compared to the DMSO-treated controls (FIG. 9b). In contrast, rapamycin-treated larvae displayed normal habituation, which did not differ significantly from the DMSO controls and was elevated compared to habituation in the FK506-treated larvae. Startle responses were slightly elevated after treatment with proINDY (FIG. 9c). CsA and FK506 treatment led to an increase in excitability, compared to excitability in the DMSO controls (FIG. 9d). Rapamycin treatment also increased excitability in comparison to the DMSO controls, although the level of excitability was lower than observed in the FK506-treated larvae. The effects of CsA and FK506 on larval excitability could not be rescued by co-treatment with proINDY. In fact, co-treatment of FK506 and proINDY led to a large increase in excitability, which was higher than the excitability observed with either compound alone.

Based on the effects of CsA and FK506, we conclude that habituation, startle responses and excitability are regulated by calcineurin signaling. However, the adverse effect of proINDY on FK506-induced hyperexcitability is not easily explained by calcineurin-NFAT signaling and may suggest the involvement of other calcineurin signaling pathways.

### Recovery of Behavior at 6 and 7 Dpf

Zebrafish larvae were treated and imaged at 5 dpf, rinsed, grown in egg water, and imaged again at 6 and 7 dpf to assess the recovery of behavior. To evaluate the effects of five single treatments on multiple behaviors at 5, 6 and 7 dpf, we calculated treatment-induced changes as compared to the DMSO control and color coded the differences in behavior (FIG. 10). The color-coded figure provides a summary of all experiments at 5 dpf, highlighting the observed changes in behavior at the time of initial imaging. CsA and FK506 treatments increased activity, decreased optomotor responses and increased excitability. In contrast, proINDY treatments decreased activity, increased optomotor responses and increased excitability. A few treatments showed a 6 dpf withdrawal effect, where changes in behavior were opposite to the changes in behavior at 5 dpf. For example, in the CsA treatment group activity was elevated at 5 dpf and decreased at 6 dpf. In the same treatment group, the optomotor response to fast red lines was suppressed at 5 dpf and elevated at 6 dpf. The summary figure shows that most, but not all, behaviors recovered in two days after treatment.

### Cluster Analysis of Behavioral Profiles

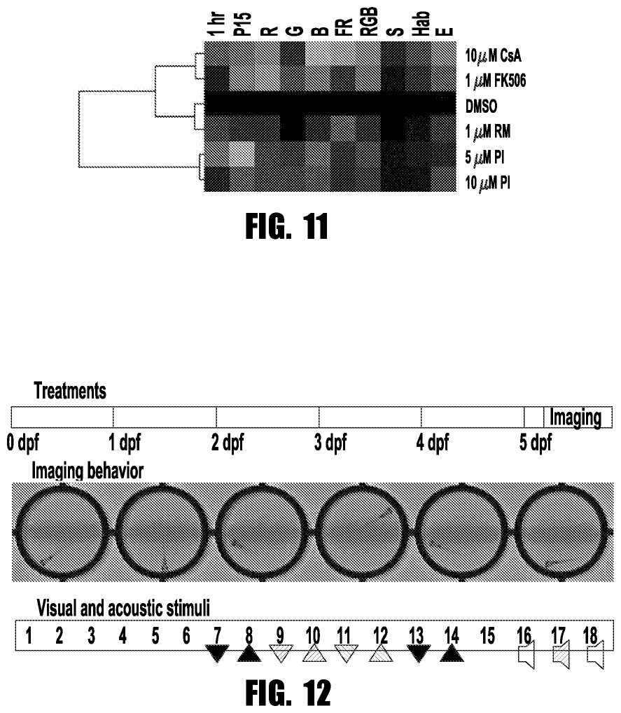

Values of multiple behaviors, as shown in FIG. 10, are often referred to as ‘behavioral profiles’ and are well suited for hierarchical cluster analysis. The cluster analysis evaluates if various treatments induce similar behavioral profiles. Cluster analysis of the 5 dpf data revealed that CsA and FK506 treatments cluster together, indicating a profile specific to calcineurin inhibition (FIG. 11). The cluster analysis has sufficient phenotypic resolution to separate the behavioral profile of FK506 from rapamycin, two macrolide immunosuppressants with similar structures but different molecular targets. Vellanki et al., Front Mol Biosci 7, 588913 (2020). Both concentrations of proINDY induce similar changes in behavior and cluster together. The proINDY behavioral profile is nearly opposite to the profile induced by inhibition of calcineurin. Based on these results, we conclude that modulators of calcineurin signaling induce specific behavioral profiles that can be grouped by cluster analyses.

### DISCUSSION

The current study shows that zebrafish larvae serve as a valuable model to study the risks and benefits of treatments that modulate calcineurin signaling. Using automated analyses of behavior, we found that the calcineurin inhibitors CsA and FK506 increase activity and decrease optomotor responses. Conversely, the DYRK inhibitor proINDY induces opposite effects, i.e. a decrease in activity and an increase in optomotor responses. These results are consistent with models of calcineurin-NFAT signaling (FIG. 5) and suggest that small-molecule treatments aimed to restore calcineurin signaling have beneficial effects on activity and visually-guided behaviors. This idea is supported by the observed rescues of behavior in co-treatments of calcineurin inhibitors and proINDY.

Some changes in behavior cannot be easily explained by the calcineurin-NFAT model. Specifically, CsA and FK506 treatments lead to an increase in acoustic excitability. These larvae continuously startle, without habituation, in response to acoustic stimuli at 1-second intervals. This behavior is not rescued by co-treatment with proINDY. Instead, such co-treatments induce an exacerbated phenotype. These results indicate that excitability may not be regulated by calcineurin-NFAT signaling and suggest the involvement of other calcineurin signaling pathways. For example, calcineurin may act by dephosphorylation of other signaling proteins such as CREB, GSK-3 and BAD. Overall, the observed hyperexcitability phenotype indicates that small-molecule treatments aimed to restore calcineurin signaling can induce adverse side effects.

Multiple measures of behavior were organized in behavioral profiles, which are suitable for hierarchical cluster analysis. Cluster analyses are typically used to examine gene expression patterns, but have also been successfully used in the analysis of behavior. Kokel et al., Nat Chem Biol 6, 231-237 (2010); Rihel et al., Science 327, 348-351 (2010). The cluster analysis performed in this study revealed a specific behavioral profile for calcineurin inhibition. In addition, the analysis had sufficient phenotypic resolution to distinguish FK506 and rapamycin, which are both macrolide immunosuppressants with similar structures, but that affect different signaling pathways.

The developed methodologies can be used to examine other previously identified DYRK and calcineurin inhibitors that may restore neural function without adverse side effects. Such inhibitors are likely to have clinical significance in various calcineurin-related disorders, including Down syndrome and Alzheimer's disease. Calcineurin and DYRK inhibitors that are not used in medicine would need to be further examined for efficacy and safety in a mammalian model system, such as the mouse, before initiating clinical trials. This route makes use of the strengths of various model systems, i.e. zebrafish are well suited for large-scale screens and mice are well suited for more detailed pre-clinical studies. In addition, a comparative approach using both zebrafish and mice can reveal fundamental mechanisms that are critical to neural function, since these core mechanisms have been conserved in the past 400 million years. A more direct bench-to-bedside approach may be possible if specific calcineurin inhibitors or DYRK inhibitors are already used in medicine or are natural products that are part of our diet. In these cases, human population studies could reveal beneficial effects in neural function, similar to the beneficial effects of CsA and FK506 in the prevention of Alzheimer's disease.

### Materials and Methods

### Zebrafish

The research project has been conducted in accordance with local and federal guidelines for ethical and humane use of animals and has been reviewed and approved by the Brown University Institutional Animal Care and Use Committee. Zebrafish (Danio rerio) embryos were collected and grown to larval stages as previously described. Thorn et al., Sci Rep 9, 13989 (2019). Adult wild-type zebrafish are maintained at Brown University as a genetically-diverse outbred strain in a mixed male and female population. Zebrafish embryos from 0-3 days post-fertilization (dpf) and zebrafish larvae from 3-5 dpf were maintained at 28.5° C. in 2 L culture trays with egg water, containing 60 mg/L sea salt (Instant Ocean) and 0.25 mg/L methylene blue in deionized water. Embryos and larvae were kept on a 12 hour light/12 hour dark cycle and were randomly assigned to different experimental groups prior to experimental manipulation. The sex of embryos and larvae cannot be determined at such early stages because zebrafish use elusive polygenic factors for sex determination, and both males and females have juvenile ovaries between 2.5 and 4 weeks of development. Zebrafish larvae were imaged at 5 dpf when the larvae display a range of locomotor behaviors and consume nutrients available in the yolk sac. Larvae are approximately 4 mm long at the 5 dpf stage.

### Pharmacological Treatments

Cyclosporine (cyclosporin A, Enzo Life Sciences), FK506 (tacrolimus, Enzo Life Sciences), rapamycin (Santa Cruz Biotechnology) and proINDY (Tocris Bioscience) were diluted in egg water from 1000× stocks dissolved in dimethyl sulfoxide (DMSO). DMSO (1 μl/ml DMSO) was added to the single treatments and the corresponding DMSO concentration was used as a vehicle control for all solutions. Larvae were exposed at 5 dpf to treatment solutions or DMSO for a total of 6 hours. Larvae were first treated in a Petri dish for 2 hours, transferred with the treatment solution to white 96-well ProxiPlates (PerkinElmer, 6006290) for 1 hour, and then imaged in the treatment solution for 3 hours. Immediately after exposure, larvae from each treatment group were washed in egg water and transferred to Petri dishes with 50 mL egg water. Larvae that were imaged again at 6 and 7 dpf were given food twice prior to each re-imaging session.

### Imaging System

Zebrafish larvae were imaged in an imaging system that holds four 96-well plates for automated analysis of behavior in a 384-well format as previously described. Briefly, the imaging system is housed in a 28.5° C. temperature-controlled cabinet where larvae in white 96-well ProxiPlates are placed onto a glass stage. Above the stage, a high-resolution camera (18-megapixel Canon EOS Rebel T6 with an EF-S 55-250 mm f/4.0-5.6 IS zoom lens) captures an image of the larvae in the four 96-well plates every 6 seconds. The camera is connected to a continuous power supply (Canon ACK-E10 AC Adapter) and controlled by a laptop computer using Canon's Remote Capture software (EOS Utility, version 3), which is included with the camera. Unlike previous descriptions of this imaging system, two small speakers (OfficeTec USB Computer Speakers Compact 2.0 System) were attached speaker-side down to the glass stage. Speakers were connected by USB to the laptop computer and set to maximum volume. Below the glass stage, a M5 LED pico projector (Aaxa Technologies) with a 900 lumens LED light source displays Microsoft PowerPoint presentations through the opaque bottom of the 96-well plates.

### Behavioral Assay

Visual and acoustic stimuli are controlled by an automated 3-hour PowerPoint presentation that is shown to the larvae. The entire 3-hour presentation has a light gray background and starts with a 1-hour period without visual or acoustic stimuli, followed by 80 minutes of visual stimuli, a 10-minute period without visual or acoustic stimuli, and 30 minutes with acoustic stimuli (FIG. 3). Larvae were not exposed to visual stimuli and acoustic stimuli at the same time.

The visual stimuli consisted of a series of moving lines that were red, green or blue. Prior studies have shown that zebrafish larvae will swim in the same direction as moving lines, a behavior that is called an optomotor response or OMR. Our previously-developed assays for visually-guided behaviors indicate 5 dpf larvae consistently respond to 1 mm thick lines set 7 mm apart that move 7 mm per 8 seconds downwards or upwards, alternating direction in 10-minute periods. Additionally, the presentation included red lines that moved at a faster speed of 7 mm per 0.5 seconds (16× faster). We used the following sequence of moving visual stimuli in subsequent 10-minute periods: downward red lines, upward red lines, downward green lines, upward green lines, downward blue lines, upward blue lines, downward fast red lines, upward fast red lines (FIG. 3). The brightness of the background (RGB=210, 210, 210), red lines (RGB=255, 0, 0), green lines (RGB=0, 180, 0), and blue lines (RGB=0, 0, 230) in the PowerPoint presentation are carefully matched to the camera settings (ISO200, Fluorescent, F5, ⅕ exposure) for optimal color separation in the automated image analysis.

The acoustic stimuli consisted of brief sine waves or ‘pulses’ (100 ms, 400 Hz) created in Audacity as 20-second sound tracks and inserted in the PowerPoint presentation. Larvae were first exposed for 10 minutes to repeated acoustic pulses with a 20-second interval, followed by 10 minutes of repeated acoustic pulses with a 1-second interval, and 10 minutes of repeated acoustic pulses with a 20-second interval.

### Image Analysis

We previously created an ImageJ macro (version 26rc091018) for automated analysis of behavior in a 384-well format (12). This macro has since been optimized for the analysis of brighter images (version 26rc062019). The ImageJ macro can analyze up to four 96-well plates, with multiple treatment groups and visual stimuli that change direction, color, and speed. Users are prompted to enter information about the plates and the periods with different visual stimuli. The software opens the first image, splits the color channels, and selects a channel in which the visual stimuli and background have similar intensities. Subsequent images are subtracted from each other to remove the background and highlight larvae that move. The software then applies a threshold (40-255), selects the first well, measures the area and centroid of the larva and logs the measurements in a ‘Results’ file. This process is automatically repeated for all wells in an image and all subsequent images in a series. The frequency of larval movement and the relative position of larva in each well is analyzed and included in the Results file. The Results file is then imported into a Microsoft Excel template (version 26rc040320—for 96-well plates). This template averages values for activity and vision in all experimental groups and imaging periods and displays the results in a graph (FIG. 6). Alternatively, the template averages values for activity in each experimental group per image. The ImageJ macro and MS Excel templates are available in the Supplementary Information and future updates will be posted on Brown University's central zebrafish website. The original results files and/or images will be made available upon request.

### Statistical Analyses

Statistical analyses were performed in MS Excel as described previously. Thorn et al., Sci Rep 9, 13989 (2019). Briefly, measurements of larval movement and position were averaged in each well during 10-minute periods. The 10-minute values were then averaged between larvae in the same treatment group. Differences between experimental groups and the corresponding controls were tested for significance. We used the non-parametric Chi-square test, since larval behaviors do not follow a normal distribution. Differences in behavior were considered significant when p<0.05, p<0.01 or p<0.001 with a Bonferroni correction for multiple comparisons (p<0.05/14, p<0.01/14 or p<0.001/14). The following 14 comparisons were made: single treatments vs. DMSO (5 comparisons); rapamycin (Rap) vs. FK506 to examine target specificity (1 comparison); CsA+PI vs. CsA to examine if 5 or 10 μM proINDY (PI) rescues the effect of CsA (2 comparisons); CsA+PI vs. PI to examine if CsA rescues the effect of 5 or 10 μM proINDY (2 comparisons); FK506+PI vs. FK506 to examine if 5 or 10 μM proINDY rescues the effect of FK506 (2 comparisons); and FK506+PI vs. PI to examine if FK506 rescues the effect of 5 or 10 μM proINDY (2 comparisons). The conservative Bonferroni correction helps to avoid type I errors (false positives), which is important in the analysis of large data sets. Internal vehicle controls were included in each imaging session.

### Cluster Analysis of Behavioral Profiles

Changes in larval activity, vision, startle response, habituation and excitability as compared to the DMSO vehicle controls were summarized in a ‘behavioral profile’. These behavioral profiles were generated for each compound used in this study. Similar profiles were grouped by hierarchical cluster analysis. The cluster analysis was carried out in Cluster 3.0 without filtering or adjusting data and using the Euclidian distance similarity metric with complete linkage. The clusters were shown in TreeView (version 1.1.6r4) using a spectrum from green (25% decrease) to red (25% increase).

### Example 3: Zebrafish Model for Calcineurin Signaling During Development

In the present study, zebrafish were used as a model system to examine changes in behavior caused by the modulation of calcineurin signaling during development. It was found that developmental exposures to the calcineurin inhibitors CsA and FK506 induced specific changes in behavior. Co-treatment with the DYRK inhibitor proINDY rescued a few behaviors but also induced a range of adverse side effects, including decreased activity and reduced optomotor responses to visual stimuli.

### Materials and Methods

Approval for animal studies. This project was carried out in accordance with federal regulations and guidelines for the ethical and humane use of animals and have been approved by Brown University's Institutional Animal Care and Use Committee (IACUC). The 3R and ARRIVE guidelines were followed for experimental design, randomization, criteria for exclusion, description of primary outcome measures and statistical methods. All experiments were carried out in compliance with the US National Research Council's Guide for the Care and Use of Laboratory Animals, the US Public Health Service's Policy on Humane Care and Use of Laboratory Animals, and the Guide for the Care and Use of Laboratory Animals.

Zebrafish. Adult wild type zebrafish (Danio rerio) are maintained at Brown University as a genetically-diverse outbred strain in a mixed male and female population. Zebrafish embryos were collected and grown to larval stages as described previously. Thorn et al., Sci Rep 9, 13989 (2019). Embryos from 0-3 days post-fertilization (dpf) and larvae from 3-5 dpf were kept on a 12 hour light/12 hour dark cycle at 28.5° C. in 2 L culture trays with egg water, containing 60 mg/L sea salt (Instant Ocean) and 0.25 mg/L methylene blue in deionized water. Zebrafish embryos and larvae were randomly assigned to different experimental groups prior to experimental manipulation. The sex of embryos and larvae cannot be determined at such early stages because zebrafish use elusive polygenic factors for sex determination, and both males and females have juvenile ovaries between 2.5 and 4 weeks of development. Liew et al., Brief Funct Genomics 13(2), 172-187 (2014). Zebrafish larvae were imaged at 5 dpf when they are approximately 4 mm long, display a range of locomotor behaviors and consume nutrients available in the yolk sac. Clift et al., Zebrafish 11(5), 455-461 (2014).

Pharmacological treatments. Zebrafish embryos and larvae were treated with 10 μM CsA (cyclosporin A, Enzo Life Sciences), 1 μM FK506 (tacrolimus, Enzo Life Sciences), 1 μM rapamycin (Santa Cruz Biotechnology), 5 μM proINDY (Tocris Bioscience) or co-treatments of either 10 μM CsA+5 μM proINDY or 1 μM FK506+5 μM proINDY. Prior studies in zebrafish have shown that these concentrations did not affect larval survival, but did induce changes in larval behavior. Clift et al., Behav Brain Res, 282, 117-124 (2015). Similarly, a 10 μM CsA exposure from 1-4 dpf did not affect survival or body length in zebrafish larvae. Robinson et al., J Appl Toxicol 37(12), 1438-1447 (2017). A 10 μM proINDY group was initially included as well, but was discontinued since developmental exposures to 10 μM proINDY affected larval survival. All administered compounds were diluted in egg water from 1000× stocks dissolved in dimethyl sulfoxide (DMSO). The corresponding DMSO concentration (1 μl DMSO/ml egg water) was used as a vehicle control. Embryos and larvae were treated during one of the following developmental stages: 2-3, 3-4, or 4-5 dpf. The embryos and larvae were treated in 10 cm Petri dishes for 24 hours, rinsed three times with egg water, and then transferred to new Petri dishes with egg water. Larvae that were treated from 4-5 dpf were rinsed and kept in egg water for 3 hours prior to imaging, typically from 10 am to 1 pm. All treatment groups were imaged at 5 dpf in egg water. For imaging, larvae were transferred to white 96-well ProxiPlates (PerkinElmer, 6006290) and were imaged for 3 hours, typically from 1-4 pm (FIG. 12).

Imaging of zebrafish larvae. Zebrafish larvae were imaged in a 384-well imaging system as described previously. Thorn et al., Sci Rep 9, 13989 (2019). Briefly, four 96-well ProxiPlates were placed on a glass stage inside a temperature-controlled cabinet, set at 28.5° C. The upper shelf of the cabinet holds a high-resolution camera (18-megapixel Canon EOS Rebel T6 with an EF-S 55-250 mm f/4.0-5.6 IS zoom lens), connected to a continuous power supply (Canon ACK-E10 AC Adapter) and controlled by a laptop computer. Images are acquired using Canon's Remote Capture software (EOS Utility, version 3), which is included with the camera. Every 6 seconds, the camera acquires a high-resolution image of larvae in the microplates (FIG. 12). Two small speakers (OfficeTec USB Computer Speakers Compact 2.0 System) are located speaker-side down on the glass stage. Speakers were connected by USB to the laptop computer and set to maximum volume (85 dBA). Below the glass plate is a M5 LED pico projector (Aaxa Technologies) with a 900 lumens LED light source, which displays Microsoft PowerPoint presentations to the larvae using the white opaque bottom of the microplates as a back-illuminated screen.

Behavioral assay. Larval behaviors were imaged for 3 hours, as described previously. Thorn et al., Sci Rep 9, 13989 (2019). During this time, a PowerPoint presentation was shown to the larvae. The PowerPoint started with a light background for 60 minutes without visual or acoustic stimuli, followed by 80 minutes with visual stimuli, 10 minutes without stimuli, and 30 minutes with acoustic stimuli (FIG. 12). Larvae were not exposed to visual stimuli and acoustic stimuli at the same time. The visual stimuli consisted of a series of lines that moved upwards or downwards in the microplates, in a horizontal plane. Prior studies have shown that zebrafish larvae will swim in the same direction as the moving lines, which is referred to as an optomotor response or OMR. Thorn et al., Sci Rep 9, 13989 (2019). This study used 1 mm thick lines set 7 mm apart that move 7 mm/8 seconds downwards or upwards, alternating direction every 10 minutes. Thorn et al., Sci Rep 9, 13989 (2019). To examine the OMR in different colors, this study used lines that were red, green, or blue. In addition, this study included red lines that moved at a faster speed of 7 mm/0.5 seconds (16× faster). The sequence of visual stimuli in 10-minute periods was as follows: downward red lines, upward red lines, downward green lines, upward green lines, downward blue lines, upward blue lines, downward fast red lines, upward fast red lines (FIG. 12). The brightness of the background (RGB=210, 210, 210), red lines (RGB=255, 0, 0), green lines (RGB=0, 180, 0), and blue lines (RGB=0, 0, 230) in the PowerPoint presentation was carefully matched to the camera settings (ISO200, Fluorescent, F5, ⅕ exposure) for optimal color separation in the automated image analysis. Larvae were exposed to acoustic stimuli (100 ms pulses at 400 Hz and 85 decibel) in the following sequence: 1) 10 minutes with 20-second intervals between pulses, 2) 10 minutes with 1-second intervals between pulses, and 3) 10 minutes with 20-second intervals between pulses. Prior studies have shown that larvae display repeated startle responses to acoustic stimuli that are 20 seconds apart, but habituate to acoustic stimuli that are 1 second apart. Tucker Edmister et al., Behav Brain Res 416, 113544 (2022). The PowerPoint presentation with moving lines and acoustic stimuli is available in a previous publication. Tucker Edmister et al., Behav Brain Res 416, 113544 (2022).

Image analysis. The inventors previously developed an ImageJ macro (version 26rc091018) for automated analysis of larval zebrafish behavior in up to four 96-well plates and multiple treatment groups. Thorn et al., Sci Rep 9, 13989 (2019). This macro has since been optimized for the analysis of brighter images (version 26rc062019) and is available in a previous publication. Tucker Edmister et al., Behav Brain Res 416, 113544 (2022). Users are prompted to enter information about the plates and the periods with different visual stimuli. The software opens the first image, splits the color channels, and selects a channel in which the visual stimuli and background have similar intensities (e.g., the red channel when using red visual stimuli). In the analysis of 96-well plates, subsequent images are subtracted from each other to remove the background and highlight larvae that move. The software then applies a threshold (40-255), selects the first well, measures the area and centroid of the individual larva and logs the measurements in a ‘Results’ file. This process is automatically repeated for all wells in an image and all subsequent images in a series. The macro calculates if a larva moved and calculates, after each movement, if a larva is located in the upper half of a well in a horizontal plane. This upper half in a horizontal plane corresponds to the upper half in a vertical plane when looking at an acquired image on a computer screen. The Results file of a single experiment contains approximately 10 million data points, i.e., 15 columns with information on the image, well, larval movement and larval location and 691,200 rows showing this information for each well in subsequent images (384 wells×1800 images).

Data processing and outcome measures. The Results files were processed in a MS Excel template. Tucker Edmister et al., Behav Brain Res 416, 113544 (2022). This template calculates the percentage of time that a larva moves (% move) and is located in the upper half of the well (% up) in subsequent 10 minute periods with various visual and acoustic stimuli (18 periods in 3 hours). For the optomotor response (OMR), larval locations are compared between two 10-minute periods when visual stimuli move up vs. down. Criteria for exclusion were set a priori in the Excel template. The template automatically excludes zebrafish larvae that move less than 1% of the time in a 3-hour recording. In addition, larvae that move less than 5% of the time in a 10-minute period are automatically excluded from OMR measurements during that period. Activity and OMR values are processed to examine the following 10 behaviors, which are the primary outcome measures of this study. 1) The average activity during the first hour of imaging without visual or acoustic stimuli. 2) The average activity in period 15 without visual or acoustic stimuli. 3) Habituation to acoustic stimuli at 1-second intervals, measured as the activity during the first 5 minutes minus the last 5 minutes of period 17. 4) Startle responses to acoustic stimuli at 20-second intervals, calculated as the activity during period 16 minus period 15. 5) Excitability in response to acoustic stimuli at 1-second intervals, calculated as the activity during period 17 minus period 16. 6) OMR using moving red lines, 7) OMR using moving green lines, 8) OMR using moving blue lines, 9) OMR using red lines, moving 16× faster than all other lines, and 10) combined OMR using moving lines of any color or speed. For each measure of behavior, we calculated the average values per treatment group and differences of these groups as compared to the DMSO vehicle controls in the same imaging experiment. These standardized differences are expressed in percentage points (% points). For example, when larvae are active 30% of the time in the DMSO controls and 20% of the time in a treated group, the effect of the treatment is calculated as −10% points.

Statistical analysis. Differences between experimental groups and the corresponding controls were tested for statistical significance in MS Excel 2016. A non-parametric Chi-square test was used, since larval activity and visually-guided behaviors do not follow a normal distribution. Thorn et al., Sci Rep 9, 13989 (2019). The Chi-square test examines observed and expected frequencies in different categories. For the frequency distribution, this study counted the number of larvae with negative % points and the number of larvae with positive % points, for each behavior in each treatment group (N=number of larvae). A Bonferroni correction was applied for multiple comparisons. This conservative correction helps to avoid type I errors (false positives), which is important in the analysis of large data sets. The following 9 comparisons were made: treated vs. DMSO (6 comparisons); rapamycin vs. FK506 to examine target specificity; CsA+proINDY vs. CsA to examine if proINDY (PI) rescues the effect of CsA; and FK506+proINDY vs. FK506 to examine if proINDY rescues the effect of FK506. Differences between experimental groups were considered significant when p<5.6×10⁻³ (0.05/9), p<1.1×10⁻³ (0.01/9), or p<1.1×10⁻⁴ (0.001/9).

### Results

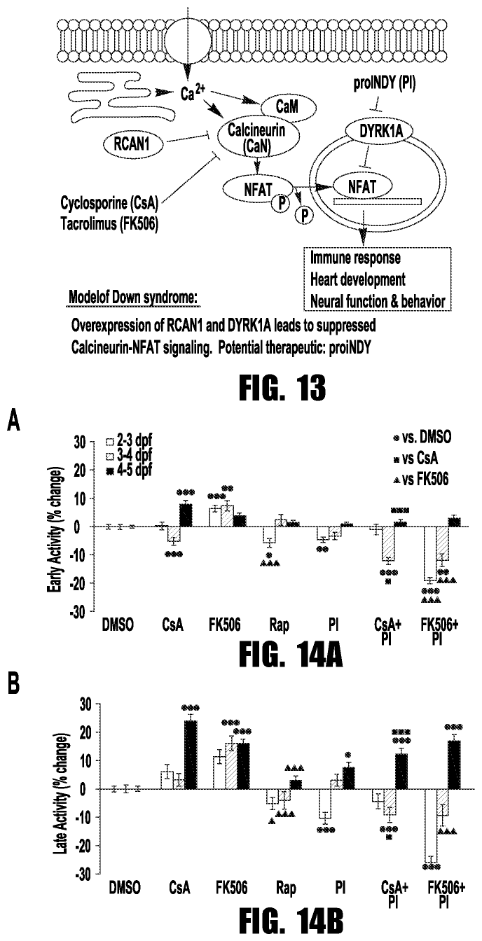

Modulation of calcineurin signaling during development. Zebrafish embryos and larvae were treated with various modulators of calcineurin signaling from 2-3, 3-4, and 4-5 dpf (FIG. 12). Based on prior studies with acute exposures. Tucker Edmister et al., Behav Brain Res 416, 113544 (2022). The following concentrations and treatment groups for the developmental exposures were selected: 1 μl/ml DMSO, 10 μM cyclosporine A (CsA), 1 μM tacrolimus (FK506), 1 μM rapamycin, 5 μM proINDY, a combination of 10 μM CsA+5 μM proINDY, and a combination of 1 μM FK506+5 μM proINDY. DMSO is used as a vehicle control and rapamycin is used as a control for target specificity. Rapamycin and FK506 are both macrolide immunosuppressants with similar structures; however, rapamycin affects Target of Rapamycin (TOR) signaling instead of calcineurin signaling. Vellanki et al., Front Mol Biosci 7, 588913 (2020). CsA and FK506 are calcineurin inhibitors and proINDY is a DYRK inhibitor, which activates the calcineurin-NFAT signaling pathway (FIG. 13). In the prior acute-exposure experiments, the inventors found that co-treatment with proINDY rescues most CsA and FK506-induced changes in behavior. Tucker Edmister et al., Behav Brain Res 416, 113544 (2022). In the current study, the inventors examined if similar results can be obtained when exposing embryos and larvae during development.

Early activity. Larval activity was examined at 5 dpf during the first hour of imaging (early activity), without visual or acoustic stimuli (FIG. 14, element A). The calcineurin inhibitors CsA and FK506 had variable effects on early activity, depending on the exposure period. For example, CsA exposures from 3-4 dpf decreased activity, while CsA exposures from 4-5 dpf increased activity, as compared to the DMSO controls. ProINDY had some beneficial effects in the co-treatments with calcineurin inhibitors. Hyperactivity induced by the 4-5 dpf exposure to CsA was effectively rescued by co-treatment with proINDY. Similarly, hyperactivity induced by 2-3 and 3-4 dpf exposures to FK506 was rescued by co-treatment with proINDY. However, proINDY also induced some adverse effects in the co-treatments. Co-treatment of CsA and proINDY from 3-4 dpf led to decreased activity levels as compared to the DMSO controls. Similarly, co-treatments of FK506 and proINDY from 2-3 and 3-4 dpf led to decreased activity levels, as compared to the DMSO controls.

Late activity. Larval activity was examined during period 15 (late activity), again without visual or acoustic stimuli (FIG. 14, element B). CsA exposure from 4-5 dpf and FK506 exposures from 3-4 and 4-5 dpf induced substantial increases in activity. Rapamycin did not induce such increases in activity, suggesting that the FK506-induced hyperactivity is specifically induced via inhibition of calcineurin signaling. ProINDY had variable effects on late activity. A 2-3 dpf proINDY exposure decreased activity, while a 4-5 dpf exposure increased activity, as compared to the DMSO controls. Co-treatment of proINDY with CsA again had some beneficial effects. Hyperactivity induced by a 4-5 dpf CsA exposure was partially rescued by co-treatment with proINDY. Similarly, hyperactivity induced by a 3-4 dpf FK506 exposure was rescued by co-treatment with proINDY. However, adverse effects were observed as well. Larvae displayed low activity after co-treatment of CsA and proINDY from 3-4 dpf and displayed low activity after co-treatment of FK506 and proINDY from 2-3 dpf. Based on these results, we conclude that modulators of calcineurin signaling can have various effects on activity depending on the developmental exposure period.

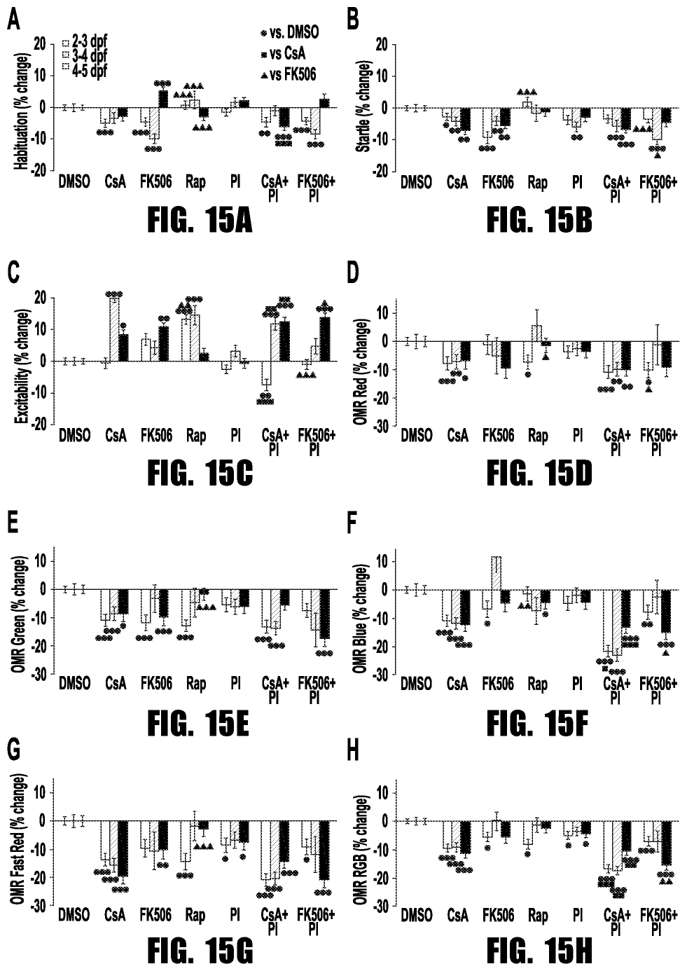

Habituation. Larval habituation to acoustic stimuli was measured using sound pulses with a 1 second interval (FIG. 15, element A). Previous studies have shown that zebrafish larvae initially display a startle response, but quickly habituate to these stimuli. Tucker Edmister et al., Behav Brain Res 416, 113544 (2022). CsA exposures from 2-3 dpf and FK506 exposures from 2-3 and 3-4 dpf reduced larval habituation to these acoustic stimuli. These larvae continue to startle in response to the 1-second acoustic stimuli. In contrast, exposure to FK506 from 4-5 dpf increased larval habituation. Co-treatment of proINDY with CsA from 4-5 dpf adversely affected habituation.

Startle responses. Startle behaviors in response to acoustic stimuli were measured with a 20-second interval (FIG. 15, element B). CsA and FK506 consistently decrease startle responses in all exposure groups, as compared to the DMSO controls. ProINDY treatments from 3-4 dpf also decrease startle responses. ProINDY has a beneficial effect in the co-treatment with FK506 from 2-3 dpf. The startle responses are strongly reduced with FK506 alone, and are largely restored in the co-treatment with proINDY. In contrast, proINDY has an adverse effect in the co-treatment with FK506 from 3-4 dpf. In this case, the startle response is reduced with FK506 alone, and is further reduced in the co-treatment with proINDY.

Excitability. The Inventors previously found that acute CsA and FK506 treatments induce hyperactivity in response to acoustic stimuli with a 1-second interval, a behavior that we refer to as excitability. Tucker Edmister et al., Behav Brain Res 416, 113544 (2022). In the present study, effects of developmental exposures on excitability were examined (FIG. 15, element C). This study found similar increases in excitability after developmental CsA exposures from 3-4 and 4-5 dpf, and developmental FK506 exposures from 4-5 dpf. ProINDY has both adverse and beneficial effects in the co-treatments with CsA and FK506, depending on the developmental period of exposure.

OMR Red. Zebrafish larvae tend to swim in the same direction as a series of moving lines, which is referred to as an optomotor response or OMR. Thorn et al., Sci Rep 9, 13989 (2019). The response to moving red lines, which were call ‘OMR Red’, were examined (FIG. 15, element D). It was found that CsA treatments decrease OMR Red in all developmental exposures. Co-treatment of proINDY and CsA induced similar decreases in OMR Red. Co-treatment of proINDY with FK506 from 2-3 dpf induced a stronger decrease in OMR Red than FK506 alone, suggesting that proINDY has an adverse effect in the co-treatment.

OMR Green. The response to moving green lines, or ‘OMR Green’, were examined (FIG. 15, element E). CsA induced a decrease in OMR Green in all developmental exposures. FK506 induced a decrease in OMR Green in the 2-3 and 4-5 dpf exposures. Similar decreases were observed in the co-treatments of proINDY with CsA and FK506, suggesting that proINDY does not induce adverse or beneficial effects in the co-treatments.

OMR Blue. The response to moving blue lines, or ‘OMR Blue’, were also examined (FIG. 15, element F). CsA induced a decrease in OMR Blue in all developmental exposure groups. FK506 induced a decrease in OMR Blue in the 2-3 dpf exposures. ProINDY induced an adverse effect in the co-treatments with CsA and FK506. The co-treatments induced stronger decreases in OMR Blue than the treatments with CsA or FK506 alone.

OMR Fast Red. Larval responses to red lines that move 16× faster than all other lines were examined (FIG. 15, element G). CsA induced a decrease in OMR Fast Red in all developmental exposure groups, as compared to the corresponding DMSO controls. FK506 induced a decrease in OMR Fast Red in the 4-5 dpf exposures. Co-treatments with proINDY induced similar decreases in OMR Fast Red, suggesting that proINDY does not induce adverse or beneficial effects in the co-treatments.

OMR RGB. This study examined larval responses to moving lines of any color or speed, by averaging all OMRs above (FIG. 15, element H). This analysis can reveal significant differences between treatment groups that did not reach statistical significance in the analysis of individual optomotor responses. CsA induced a decrease in OMR RGB in all developmental exposure groups. FK506 induced a decrease in OMR RGB in the 2-3 dpf exposure group. ProINDY induced a decrease in OMR RGB in the 2-3 and 4-5 dpf exposure groups. Co-treatment of proINDY and CsA had an adverse effect on OMR RGB in all developmental exposure groups. Similarly, co-treatment of proINDY and FK506 had an adverse effect on OMR RGB in the 4-5 dpf exposure group.

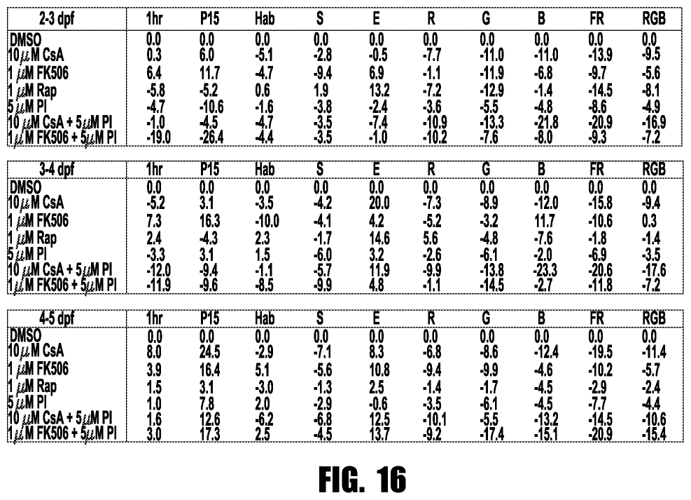

Behavioral profiles. The observed patterns of behavior were summarized in color-coded behavioral profiles (FIG. 16). These profiles provide an overview of the changes in behavior induced by the calcineurin inhibitors, proINDY, and the co-treatments. For example, treatment with the calcineurin inhibitors CsA and FK506 from 2-3 dpf induces hyperactivity, low habituation, and low optomotor responses, which is not observed in any other 2-3 dpf treatment group. The behavioral profiles are different for FK506 and rapamycin, both of which are macrolide immunosuppressants with similar structures but different molecular targets. Vellanki et al., Front Mol Biosci 7, 588913 (2020). In FIG. 16, the deep colors (dark green and dark red) in the co-treatment groups indicate that proINDY does not effectively rescue the CsA and FK506-induced changes in behavior.

Based on these results, it was concluded that inhibition of calcineurin signaling during development induces specific changes in behavior. ProINDY can restore normal behaviors, either partially or completely, when larvae are co-treated with proINDY and calcineurin inhibitors. However, proINDY can also exacerbate changes in behavior in these co-treatments. Thus, proINDY can have either beneficial or adverse effects when calcineurin signaling is inhibited, depending on the developmental exposure period and the specific behaviors that are examined.

### DISCUSSION

The present study shows that inhibition of calcineurin signaling during development leads to specific changes in zebrafish larval behavior. Larvae displayed hyperactivity and suppressed optomotor responses in all developmental exposure groups (2-3, 3-4, or 4-5 dpf). The suppressed visually-guided behaviors are consistent with a prior study in zebrafish showing that CsA treatment from 2-3 dpf decreased the response to a moving red bar in 5-lane plates. Clift et al., Behav Brain Res, 282, 117-124 (2015). In a recent study, the Inventors examined acute exposures at 5 dpf starting 3 hours before imaging and found that both CsA and FK506 induced hyperactivity and decreased optomotor responses. Tucker Edmister et al., Behav Brain Res 416, 113544 (2022). Thus, the developmental and acute exposures to calcineurin inhibitors had similar effects on behavior. In the latter study, acute treatments with proINDY led to low activity and increased optomotor responses. Thus, proINDY and calcineurin inhibitors had opposing effects. In addition, proINDY effectively rescued most CsA and FK506-induced changes in behavior. In the current study, proINDY had more variable effects on behavior, depending on the exposure period during development. For example, proINDY treatment from 2-3 dpf decreased activity, while proINDY treatment from 4-5 dpf increased activity. In addition, proINDY had both beneficial and adverse effects in the co-treatments with calcineurin inhibitors. The variable effects of proINDY during development may be explained by multiple signaling pathways that act during development. A key pathway is the calcineurin-NFAT signaling pathway, which is activated by proINDY via the inhibition of an inhibitor (FIG. 13). However, calcineurin may also act by dephosphorylating other signaling proteins such as CREB, GSK-3 and BAD. Reese et al., Curr Neuropharmocol, 9(4), 685-692 (2011). These signaling pathways are suppressed by calcineurin inhibitors, but may not be directly affected by proINDY.

Small molecule DYRK inhibitors have been proposed as potential therapeutics for Down syndrome. Jarhad et al., J Med Chem, 61(22), 9791-9810 (2018). The basic idea is that the suppressed calcineurin-NFAT signaling pathway may be rescued by activation of NFAT (FIG. 13). However, this study's results suggest that the DYRK inhibitor proINDY has substantial risks and limited benefits when used during development. It is proposed that key questions on calcineurin signaling during brain development need to be pursued before initiating clinical trials. For example, which DYRK inhibitors are effective and safe? What are the sensitive periods during development? Does calcineurin act via NFAT, CREB, GSK-3, BAD, or other proteins in the developing brain? Should studies aim to activate calcineurin, instead of NFAT? Do other Down syndrome-related proteins, such as Down syndrome cell adhesion molecules (Ma et al., J Neurosci, 40(1), 143-158 (2020)), play a role in neural function?

The zebrafish model will be useful in answering some of these questions. The current study shows that the automated analysis of zebrafish larval behavior can be used to examine both beneficial and adverse effects of calcineurin modulation during development. In addition, acute exposures may be used in small molecule screens to identify novel modulators of calcineurin signaling.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while various theories are presented describing possible mechanisms through with the compounds are effective, the compounds are effective regardless of the particular mechanism employed and the inventors are therefore not bound by theories described herein. The invention is not limited to the exact details shown and described, for variations obvious to one skilled in the art will be included within the invention defined by the claims.

What is claimed is:

1. A method of treating or preventing a neurodegenerative disease or disorder, comprising administering a therapeutically effective amount of a CsA-type drug to a subject in need thereof.

2. The method of claim 1, wherein the neurodegenerative disease is Alzheimer's disease.

3. The method of claim 1, wherein the CsA-type drug is selected from the group of drugs consisting of bromocriptine, tetrabenazine, rosiglitazone, nebivolol, sorafenib, XL184, tamoxifen, meclizine, salmeterol xinafoate, sulfasalazine, irbesartan, flutamide, celecoxib, and cabergoline.

4. The method of claim 1, wherein the CsA-type drug is XL184.

5. The method of claim 1, wherein the CsA-type drug is used to treat a neurodegenerative disease or disorder.

6. The method of claim 1, wherein the CsA-type drug is used to prevent a neurodegenerative disease or disorder.

7. The method of claim 1, wherein the subject is a human subject.

8. The method of claim 1, wherein the method further comprises administering a therapeutically effective amount of an inhibitor DYRK (INDY) type drug to the subject.

9. A method of treating Down syndrome, comprising administering a therapeutically effective amount of an inhibitor DYRK (INDY) type drug to a subject in need thereof.

10. The method of claim 9, wherein the INDY type drug is selected from the group consisting of Lapatinib, Bazedoxifene, Rucaparib, Ibutilide, Clotrimazole, Duloxetine, Tranylcypromine, Tizanidine, Venlafaxine, and UK 14,304.

11. A method of identifying a neuromodulating drug, comprising:
contacting a zebrafish larvae with a test drug;
stimulating the zebrafish larvae with light and/or sound;
observing the activity of the zebrafish in response to the stimulation; and
characterizing the test drug as a neuromodulating drug if the activity of the zebrafish indicates an effect of the test drug on calcineurin or DYRK signaling in the zebrafish larvae.

12. The method of claim 11, wherein the method comprises identifying a CsA-type drug.

13. The method of claim 11, wherein the method comprises identifying an INDY-type drug.

14. The method of claim 11, wherein a plurality of zebrafish larvae are contacted with the test drug in a multi-well plate.

15. The method of claim 11, wherein the activity is locomotor activity.

16. The method of claim 11, wherein the activity comprises multiple behaviors, and characterizing the activity comprises cluster analysis.
